(19) United States
(12) Patent Application Publication
(10) Pub. No.: US 2024/0342448 A1
(43) Pub. Date: Oct. 17, 2024

(54) ACCESS DEVICE, INTRODUCER FOR ACCESS DEVICE, AND METHODS OF USE

(71) Applicant: ATROPOS LIMITED, Bray (IE)

(72) Inventors: Stephen WILLIAMS, Co. Wicklow (IE); Joseph HYLAND, County Dublin (IE); Frank BONADIO, County Wicklow (IE); Shane MACNALLY, County Wicklow (IE); Olwen COUGHLAN, Dublin 18 (IE)

(73) Assignee: ATROPOS LIMITED, Bray (IE)

(21) Appl. No.: 18/634,615

(22) Filed: Apr. 12, 2024

Related U.S. Application Data
(60) Provisional application No. 63/579,875, filed on Aug. 31, 2023.
(60) Provisional application No. 63/495,898, filed on Apr. 13, 2023.

(51) Int. Cl. A61M 29/00

(52) U.S. Cl. CPC A61M 29/00

(57) ABSTRACT

An introducer for inserting a medical access device into an opening of a patient, the medical access device including a sleeve portion including a proximal end and a distal end; a proximal ring coupled to a proximal end of the sleeve portion; a collapsible distal anchor member coupled to the distal end of the sleeve portion; wherein the sleeve portion, proximal ring, and distal anchor member form a lumen for access into the opening of the patient, the introducer including: a shaft having a proximal end and a distal end; and an introducer head at the distal end of the shaft, the introducer head including a plurality of distally-extending prongs.

### CROSS-REFERENCE TO RELATED APPLICATIONS

This application claims the benefit of priority of U.S. Provisional Patent Application No. 63/495,898, filed Apr. 13, 2023 and U.S. Provisional Patent Application No. 63/579,875, filed Aug. 31, 2023, the entirety of all of which are incorporated herein by reference.

### TECHNICAL FIELD

The present disclosure relates generally to medical systems and devices for accessing a body cavity, and more particularly, to methods and devices for transvaginal access during a medical procedure.

### BACKGROUND

Medical devices are used within body cavities to perform a variety of medical procedures. The body cavities may be accessed through openings in a body including a natural orifice such as a transvaginal opening, anus or via an incision. Procedures occurring in a body cavity accessed via an opening in a body, e.g., a natural orifice such as a transvaginal opening, anus or via an incision, may require a device to assist in accessing the body cavity. It may be desirable to ensure that the devices assisting in accessing the body cavity are securely inserted and attached into the openings of a body.

This disclosure may address one or more of these problems or other problems in the art. The scope of the disclosure, however, is defined by the attached claims and not the ability to address a specific problem.

### SUMMARY OF THE DISCLOSURE

According to an aspect, an introducer for inserting a medical access device into an opening of a patient, the medical access device including a sleeve portion including a proximal end and a distal end; a proximal ring coupled to a proximal end of the sleeve portion; a collapsible distal anchor member coupled to the distal end of the sleeve portion; wherein the sleeve portion, proximal ring, and distal anchor member form a lumen for access into the opening of the patient, the introducer including: a shaft having a proximal end and a distal end; and an introducer head at the distal end of the shaft, the introducer head including a plurality of distally-extending prongs.

In some aspect, the techniques described herein relate to an introducer, wherein the distally-extending prongs include a first prong and a second prong located at a distal most end of the introducer head.

In some aspect, the techniques described herein relate to an introducer, wherein the first prong includes an inner concave surface, and the second prong includes an inner concave surface, and inner concave surfaces of the first and second prongs face one another.

In some aspect, the techniques described herein relate to an introducer, wherein the introducer head includes a third prong in between the first prong and the second prong at the distal most end of the introducer head.

In some aspect, the techniques described herein relate to an introducer, wherein the first prong has a greater length than the second prong, and both the first prong and second prong are curved in a same direction.

In some aspect, the techniques described herein relate to an introducer, wherein the plurality of distally-extending prongs of the introducer head form a fork shape.

In some aspect, the techniques described herein relate to an introducer, wherein the introducer consists essentially of the shaft and the fork-shaped introducer head.

According to an aspect, a medical access device including an access device for access through an opening of a patient, and an introducer for the access device, the access device including: a sleeve portion including a proximal end and a distal end; a proximal ring coupled to a proximal end of the sleeve portion; and a collapsible distal anchor member, coupled to the distal end of the sleeve portion; wherein the sleeve portion, proximal ring, and distal anchor member form a lumen for access through an opening of a patient, and the introducer including: a shaft having a proximal end and a distal end; and a fork-shaped distal end portion at the distal end of the shaft configured to receive and deploy the collapsible distal anchor member.

In some aspect, the techniques described herein relate to a medical access device, wherein the collapsible distal anchor member includes a plurality of hinge points.

In some aspect, the techniques described herein relate to a medical access device, wherein each of the plurality of hinge points is a semi-circular protrusion that extends radially outwards, wherein a radius of the semi-circular protrusion is smaller than a radius of the collapsible distal anchor member.

In some aspect, the techniques described herein relate to a medical access device, wherein the plurality of hinge points includes a first hinge point, a second hinge point, a third hinge point, and a fourth hinge point, wherein the first hinge point and second hinge point are one hundred and eighty degrees apart; the third hinge point and fourth hinge point are one hundred and eighty degrees apart; and the first hinge point and third hinge point are ninety degrees apart.

In some aspect, the techniques described herein relate to a medical access device, wherein the collapsible distal anchor member is one of a: partially exposed nitinol ring; a concertina ring; a severed ring; a spring ring; a sliding ring; a concertina section ring; or a notched ring.

In some aspect, the techniques described herein relate to a medical access device, wherein the a fork-shaped distal end portion include an introducer head with a first prong and a second prong located at a distal most end of the introducer head.

In some aspect, the techniques described herein relate to a medical access device, wherein the first prong includes an inner concave surface, and the second prong includes an inner concave surface, and inner concave surfaces of the first and second prongs match a curvature of an outer surface of the collapsible distal anchor member.

In some aspect, the techniques described herein relate to a medical access device, wherein the shaft has a length from the proximal end to the distal end of 5 cm to 30 cm.

In some aspect, the techniques described herein relate to a medical access device, the opening of the patient includes a body opening.

According to an aspect, a method of inserting an access device into a body cavity, the access device including a sleeve portion including a proximal end and a distal end; a proximal ring coupled to a proximal end of the sleeve portion; a collapsible distal anchor member coupled to the distal end of the sleeve portion; wherein the sleeve portion, proximal ring, and the collapsible distal anchor member form a lumen for access into the body cavity, the method including: compressing a first portion and a second portion of the collapsible distal anchor member, wherein the first portion and second portion of the collapsible distal anchor member are located opposite one another; positioning the first portion and the second portion of the collapsible distal anchor member into an introducer, the introducer including: a shaft having a proximal end and a distal end; and a fork-shaped distal end portion configured for securing and deploying the collapsible distal anchor member; folding a third portion and a fourth portion of collapsible distal anchor member down the shaft of the introducer towards the proximal end; inserting the distal end portion of the introducer into the body cavity; releasing the collapsible distal anchor member within the body cavity by retracting the introducer; and extracting the introducer from the body cavity.

In some aspect, the techniques described herein relate to a method, further including, upon extracting the introducer from the body cavity: expanding an outer wall of the sleeve portion so that a cavity wall of the body cavity retracts; and inserting one or more medical instruments through the access device and into the body cavity.

In some aspect, the techniques described herein relate to a method, wherein positioning the collapsible distal anchor member into an introducer includes placing the compressed first portion and the second portion of the collapsible distal anchor member within prongs of the distal end portion of the introducer.

In some aspect, the techniques described herein relate to a method, wherein releasing the collapsible distal anchor member within the body cavity includes the distal anchor member sliding off the fork-shaped distal end portion and expanding radially in the body cavity.

### BRIEF DESCRIPTION OF THE DRAWINGS

FIGS. 1A-1H illustrates a collapsible wire ring as a possible component of a medical device, according to an example;

FIGS. 2A-2E illustrates a collapsible wire ring with tubes as a possible component of a medical device, according to an example;

FIGS. 3A-3D illustrates a concertina ring device as a possible component of a medical device, according to an example;

FIGS. 4A-4B shows concertina ring device inserted into and through a vagina

FIGS. 5A-5B shows concertina ring device inserted into the inside of containment bag situated in the body cavity, according to an example;

FIGS. 6A-6G show a severed ring medical device, according to an example;

FIGS. 7A-7C illustrate that which is shown and described in FIGS. 6E-6G but within a vagina and through colpotomy into a body cavity

FIGS. 8A-8C repeats that which is described in FIGS. 7A-7C respectively but instead shows medical severed ring device inserted into the inside of containment bag situated in the body cavity and vagina, according to an example;

FIGS. 9A-9C shows a spring distal ring that may be used in any sleeved retractor medical device, according to an example;

FIGS. 10A-10C shows another ring embodiment, sliding ring device as a possible component of a medical device, according to an example;

FIGS. 11A-11C shows another ring embodiment, a concertina section ring device as a possible component of a medical device, according to an example;

FIGS. 12A-12D shows another ring embodiment, a notched ring device, according to an example;

FIGS. 13A-13D shows a selection of pronged medical device anchoring ring introducers, according to an example;

FIGS. 14A-14C shows a device introducer similar to that shown in FIGS. 13A-13D, according to an example;

FIGS. 15A-15C represent FIGS. 14A-14C respectively from a top down view for illustrative purposes

FIGS. 16A-16E expands further on this insertion example using another pronged introducer device, according to an example;

FIGS. 17A-17B further demonstrated insertion of medical device, according to an example;

FIGS. 18A-18C show another introducer embodiment, a long introducer head device, according to an example;

FIG. 19 shows another introducer device with curved introducer head, according to an example;

FIGS. 20A-20C illustrate the use of a medical device curved introducer retractor, according to an example;

FIGS. 21A-21C show the curved introducer retractor and curved intro head device demonstrated in anatomical view but with the addition of containment bag, according to an example;

FIGS. 22A-22B show a medical device hooded introducer device for deployment of a ring, according to an example;

FIGS. 23A-23B show medical device hooded introducer device with guard, according to an example;

FIGS. 24A-24C show the top view of hooded introducer device

FIGS. 25A-25D further illustrate device hooded introducer device with guard in an anatomical view

FIGS. 26A-26C further illustrate hooded introducer device with guard in another anatomical view

FIGS. 27A-27C further illustrate hooded introducer device with guard in the same anatomical view as FIGS. 26A-26C but with the inclusion of containment bag and specimen for removal, according to an example;

FIGS. 28A-28F show medical device sleeved introducer device, according to an example;

FIGS. 29A-29B show evolution of sleeved introducer device with ring to incorporate with a guarded sleeve retractor, according to an example;

FIGS. 30A-30B show perspective views of sleeved introducer device with ring

FIGS. 31A-31C show a medical device tear away introducer device, according to an example;

FIGS. 32A-32C show the top-down view of FIGS. 31A, 31B and 31C respectively, of tear away introducer device

FIGS. 33A-33B show tear away introducer device being inserted through a vagina into a body cavity

FIGS. 34A-34E illustrate a slotted tube introducer device that may be used to facilitate insertion of a medical device into a body cavity, according to an example;

FIGS. 35A-35B show slotted tube introducer device being utilised in an anatomical view

FIGS. 36A-36C shows a medical device complete with tubing introducer device, according to an example;

FIGS. 37A-37B show tubing introducer device being utilised in an anatomical view

FIGS. 38A-38C show, in isolation, guard elements with 3 different length petal skirts; long petal skirt, medium petal skirt and short petal skirt respectively, according to an example;

FIGS. 39A-39C show motion of guard forced downwards for devices long petal skirt guard, medium petal skirt guard medium petal skirt guard and short petal skirt guard respectively

FIG. 40 shows outer sheath device, according to an example;

FIGS. 41A-41B show the effective shortening of guard petal reach in the anatomy while using medium petal skirt guard in conjunction with sleeve and proximal ring

FIGS. 42A-42B show outer sheath device complete with introducer tube with funnel being inserted into a vagina, according to an example;

FIGS. 43A-43I show a standard ring 1307 in a sprung clasp introducer device, according to an example;

FIGS. 44A-44C show a medical device sprung ring holder with funnelled tube, according to an example;

FIGS. 45A-45C shows sprung ring holder funnelled tube with rigid rod introducer, according to an example;

FIGS. 46A-46C and FIGS. 47A-47C shows removable cap introducer device from front view and top view respectively, according to an example;

FIGS. 48A-48C shows an isometric view of removable introducer cap

FIGS. 49A-49C and FIG. 50 show guide tube removable cap introducer, according to an example;

FIGS. 51A-51C show medical device with removable cap introducer, according to an example;

FIGS. 52A-52C shows flexible section removable cap introducer, according to an example;

FIGS. 53A-53E show pully hood introducer, according to an example;

FIGS. 54A-54C show pully introducer medical device including a sleeve and guard elements, according to an example;

FIGS. 55A-55E illustrate pully introducer medical device being inserted into an anus

FIGS. 56A-56C shows device pully introducer medical device, by way of an example embodiment from this application, inserted through an abdominal incision into the bladder

FIGS. 57A-57C shows pully introducer medical device, by way of an example embodiment from this application, inserted through an abdominal incision into the stomach

FIGS. 58A and 58B show collapsible hinge ring including four small radius sections and four large radius sections, according to an example;

FIGS. 59A and 59B show collapsible hinge ring and its cross in a somewhat oval shape

FIGS. 60A and 60B shows round cross section ring and the effect that having a circular cross section has on the side profile when it is compressed

FIGS. 61A and 61B show the attachment of collapsible hinge ring to rounded head introducer, according to an example;

FIGS. 62A-62C show the steps described in FIGS. 61A and 61B from a top view

FIGS. 63A-63D show the step-by-step introducer of collapsible hinge ring using introducer

FIGS. 64A-64C show medical retractor device being introduced into a vagina, according to an example;

FIGS. 65A-65E show a separate guard device, according to an example;

FIG. 66A and 66B show a hinged ring with notches, similar to collapsible hinge ring, according to an example;

FIGS. 67A-67E shows a tubed distal ring according to another example.

### DETAILED DESCRIPTION

The present disclosure is now described with reference to exemplary medical access devices that may be used in accessing the interior of the body. However, it should be noted that reference to any particular procedure is provided only for convenience and not intended to limit the disclosure. A person of ordinary skill in the art would recognize that the concepts underlying the disclosed devices and application methods may be utilized in any suitable procedure, medical or otherwise. The present disclosure may be understood with reference to the following description and the appended drawings, wherein like elements are referred to with the same reference numerals.

For ease of description, portions/regions/ends of a device and/or its components are referred to as proximal and distal ends/regions. It should be noted that the term “proximal,” is intended to refer to ends/regions closer to a user of the disclosed device (e.g., outside the body of the patient), and the term “distal,” used herein to refer to ends/regions farther away from the user of the disclosed device (e.g., inside the body of the patient). Similarly, extends “distally” indicates that a component extends in a distal direction, and extends “proximally” indicates that a component extends in a proximal direction. Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

The present disclosure relates generally to medical systems and devices for accessing a body cavity through an incision or a natural orifice, and more particularly, to methods and systems for inserting these transvaginal access devices during a medical procedure.

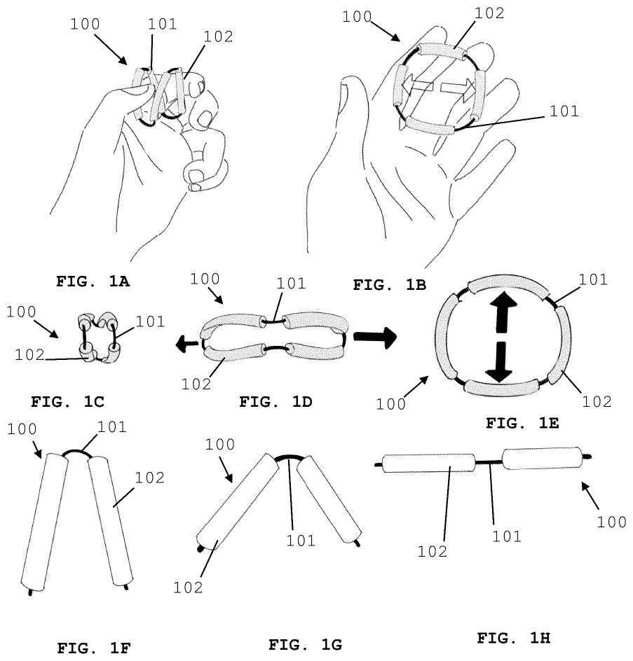

FIGS. 1A-1H illustrates a collapsible wire ring 100, according to an example, as a possible component of a medical device. The collapsible wire ring 100 may include a wire inner portion 101 and solid outer ring segments 102. The collapsible wire ring 100 may be incorporated into a medical device used for accessing the body cavity via an opening in the body. An opening in a body may refer to an incision or any natural office (e.g., a vagina or rectum) in a patient. The collapsible wire ring 100 may be used as an anchoring ring for an access system in the body cavity. The collapsible wire ring 100 may range in diameter from preferably 2 cm to 10 cm. The diameter may be sized to secure a medical device within an opening of a patient as described below.

The wire inner portion 101 of the collapsible wire ring 100 may be a flexible or super elastic or shape memory metal, for example nitinol. The wire inner portion 101 may be circular or ovular in shape. The collapsible wire ring 100 may include two or more solid outer ring segments 102. For example, the collapsible wire ring may have two, four, six, eight, or 10 solid outer ring segments 102. The solid outer ring segments 102 may be made from flexible or semi rigid plastic or rubber and have a cross-sectional diameter greater than the wire inner portion 101. The solid outer ring segments 102 may have a cross section with a hollow center configured to receive the wire inner portion 101. The solid outer ring segments 102 may circumferentially surround a portion of the wire inner portion 101.

As shown in FIGS. 1A-1H, the solid outer ring segments 102 may be arranged in such a way about wire inner portion 101 such that areas of wire inner portion 101 may be left exposed (e.g., hinge points) without coverage from solid outer ring segments 102. A hinge point may refer to an area along an anchoring member/distal ring (e.g., the collapsible wire ring 100), that is configured to fold or bend. These hinge points may assist in compressing and decompressing an anchoring member during insertion into an opening in a body. At these areas, collapsible wire ring 100 may be allowed to bend to a much smaller radius than would otherwise be possible in a ring with solid, single material and single cross section diameter construction. FIG. 1A shows the collapsible wire ring 100 being compressed by hand into a collapsed configuration by way of the highly bendable areas wire inner portion 101. FIG. 1B shows that when collapsible wire ring 100 may be released from the compression force of the hand, the super elastic or self-sprung nature of wire inner portion 101 may allow for collapsible wire ring 100 to expand to the original circular shape as indicated by the arrows. FIG. 1C shows collapsible wire ring 100 in a collapsed configuration from a top view. FIG. 1D shows collapsible wire ring 100 in a semi-collapsed configuration from a top view. FIG. 1E shows collapsible wire ring 100 in an open configuration from a top view. FIG. 1F shows collapsible wire ring 100 in a collapsed configuration from a side view. FIG. 1G shows the collapsible wire ring 100 in a semi-collapsed configuration from a side view. FIG. 1H shows the collapsible wire ring 100 in an open configuration from a side view.

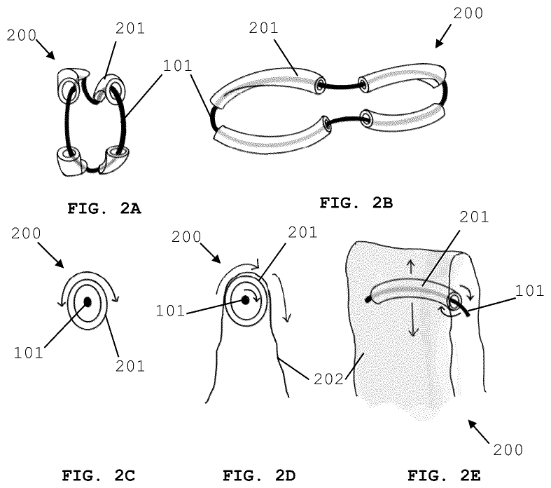

FIGS. 2A-2E illustrates a collapsible wire ring with tubes 200, according to an example, as a possible component integrated with a medical device. The collapsible wire ring with tubes 200 may be made up of a wire inner portion 101 and tubular outer ring segments 201. The collapsible wire ring with tubes 200 may be incorporated into a medical device used for accessing the body cavity via an opening in the body, e.g. via an incision or a natural orifice (e.g., a vagina or rectum). The collapsible wire ring with tubes 200 may be used as an anchoring ring for an access system in the body cavity.

The wire inner portion 101 may be a flexible or super elastic or shape memory metal, for example nitinol. The solid outer ring segments 201 may be made from flexible or semi rigid plastic or rubber and have a cross-sectional diameter greater than the wire inner portion 101. The solid outer ring segments 201 may have a cross section with a hollow center configured to receive the wire inner portion 101. The solid outer ring segments 201 may circumferentially surround a portion of the wire inner portion 101. The tubular outer ring segments 201 may be free to move along wire inner portion 101 but there still remains areas where wire inner portion 101 may be left exposed without coverage from the tubular outer ring segments 102 (these areas may be referred to as hinge points). At these hinge points, collapsible wire ring with tubes 200 may be allowed to bend to a much smaller radius than would otherwise be possible in a ring with solid, single material and single cross section diameter construction. FIGS. 2A and 2B show a top view of collapsible wire ring with tubes 200 in a collapsed configuration and semi collapsed configuration respectively. In this way the collapsible wire ring with tubes 200 may insert through a smaller diameter natural orifice or incision in an opening of a body more easily.

FIGS. 2C-2E illustrate how tubular outer ring segments 201, according to an example, may be free also to spin about wire inner portion 101 as well as sliding along the wire inner portion 101. FIG. 2C shows a cross section of collapsible wire ring with tubes 200. For example, the wire inner portion 101 may be configured to stay stationary, without rotation while solid outer ring elements may be configured to rotate both clockwise and/or counter clockwise (as indicated by the arrows) about the wire inner portion 101. FIG. 2D shows cross sectional view of collapsible wire ring with tubes 200 placed within a sleeve 202. As will be described further below, the sleeve 202 may form a portion of a guard assembly for use in medical procedure. The sleeve 202 may be a continuous piece of material that is flexible and may confirm to a shape and size of a body opening. The sleeve 202 may be a medical grade material, including a polyurethane film, a Kevlar or other cut or puncture resistant material, or the like. When sleeve 202 is pulled around collapsible wire ring with tubes 200, the friction between sleeve 202 and tubular outer ring segments 201 may cause tubular outer ring segments 201 to rotate about wire inner portion 101 (as indicated by the arrows) similar to that of a pully system. The ability of tubular outer ring segments 201 to spin about wire inner portion 101 may allow the sleeve to pass around the exterior of the collapsible wire ring with tubes 200 with minimal friction. FIG. 2E shows a perspective view of FIG. 2D. FIG. 2E shows a sleeve 202, as part of a medical device, passing around collapsible wire ring with tubes 200 freely while causing tubular outer ring segments 201 to spin about wire inner portion 101. The minimal friction passing of sleeve 202 around collapsible wire ring with tubes 200 may be of benefit in any medical device where collapsible wire ring with tubes 200 may be utilized in conjunction with a sleeve 202 and where collapsible wire ring with tubes 200 may be required to pass around tubular outer ring segments 201 with as little resistance as possible. The arrows on sleeve 202 may indicate the movement of the sleeve 202 about a circumference of the wire ring with tubes 200.

In all embodiments within this application where a distal ring or anchor ring may be utilized, the collapsible wire ring 100 and collapsible wire ring with tubes 200 may be utilized where not otherwise specified. In general, all distal rings described and/or included in this application in isolation or within various embodiments may be interchangeable across all embodiments.

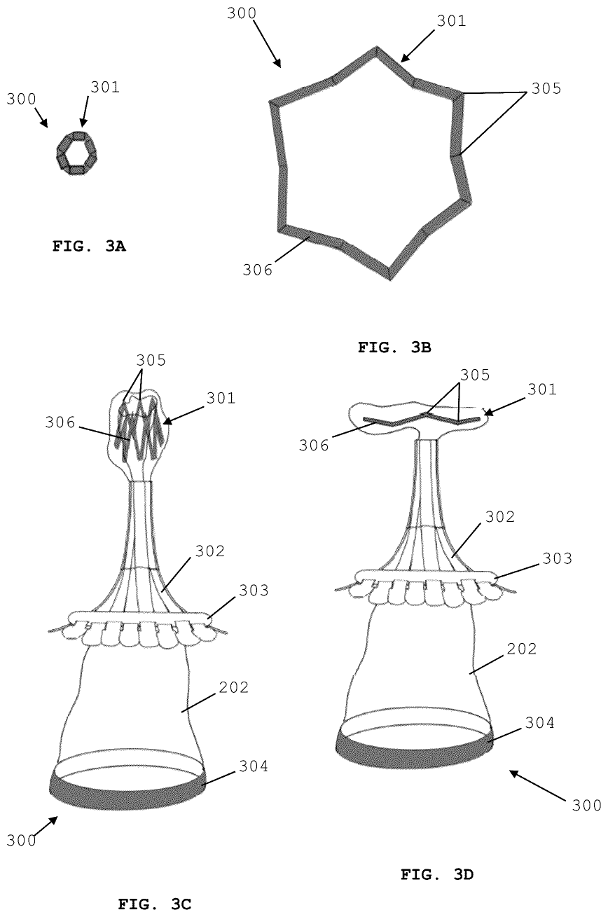

FIGS. 3A-3D illustrates a concertina ring device 300, according to an example, as a possible component of a medical device, according to an example. Concertina ring medical device 300 may include a concertina distal ring 301, guard petals 302, guard ring 303, proximal ring 304 and sleeve 202 which connects proximal ring 304 to guard petal 302. Sleeve 202 may connect proximal ring 304 to guard petal 302 by passing through the center of guard petals 302 which may be arranged circumferentially about guard ring 303. The sleeve 202 may be configured to fold backwards in order to attach to the distal region of guard petal 302. This configuration of sleeve 202 may create a pocket in the bottom of the sleeve 202 within the concertina ring medical device 300 where concertina distal ring 301 may be placed.

FIGS. 3A and 3B show a concertina distal ring 301, according to an example, in a closed (FIG. 3A) and opened configuration (FIG. 3B) respectively from a top view. Concertina distal ring 301 may have a series of inbuilt concertina hinge points 305 that may allow concertina distal ring 301 to move freely between open and closed configurations by allowing each concertina ring member 306 to rotate at its end point relative to its neighboring concertina ring member 306. The concertina ring device 300 may have two configurations, compressed as shown in FIG. 3A and uncompressed as shown in FIG. 3B. The concertina ring device 300 may be configured to be compressed prior to insertion into a body opening, and may naturally expand to the uncompressed shape upon positioning the concertina ring device 300 in a body opening. The concertina ring device 300 may include a set of concertina ring members 306. For example, there may be between eight to thirty concertina ring members 306 and corresponding hinge points 305.

FIGS. 3C and 3D show concertina distal ring 301, according to an example, incorporated into an overall concertina ring device 300 with concertina distal ring 301 shown in a compressed (FIG. 3C) and uncompressed (FIG. 3D) configuration respectively. Concertina distal ring 301 may be captured in the distal end of sleeve 202 where it doubles back on itself to connect to guard petal 302, thus creating a pocket.

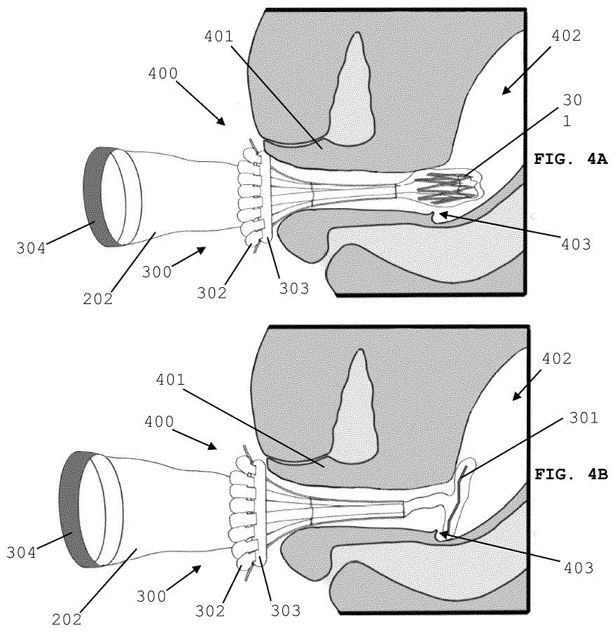

Concertina ring medical device 300 may be inserted into the body opening (e.g., a natural orifice or incision) to provide access to the body cavity. One such natural orifice may be the vagina 400 where the concertina ring device 300 would contact, line, and may retract the vaginal wall 401 as shown in FIGS. 4A and 4B. Similarly, as shown in FIGS. 4A and 4B concertina ring device 300 may be inserted into the body cavity 402 through a colpotomy 403, an incision formed when the cervix has been detached or a posterior or partial colpotomy posterior the cervix at the rear of the vagina 400, the proximal ring 304 may be positioned outside body cavity colpotomy 403 and concertina distal ring 301 may be positioned inside body cavity 402 so that a surgeon may access colpotomy 403 to perform a medical procedure.

FIG. 4A shows concertina ring medical device 300 inserted through the vagina 400 and colpotomy 403. Concertina ring medical device 300 may be designed to have one or more sizes and may fit in different sized openings. In some instances, concertina ring medical device 300 may have a larger overall diameter than vagina 400 channel and therefore may create a seal against a tissue/vaginal wall 401 surrounding the introitus of vagina 400. In some instances, the proximal ring 304 may be flipped/rolled distally in order to shorten the effective length of sleeve 202. In doing so, concertina ring device 300 may cause vaginal wall 401 surrounding the opening of vagina 400 to move radially outward (e.g. retract) and the effective length of the vaginal canal (between the introitus and cervix) to shorten. Vaginal wall 401 may be held or urged radially outward by sleeve 202. By urging outwards against the vaginal wall 401, concertina ring device 300 may create a seal in the vagina 400 to prevent fluid or materials from escaping colpotomy 403 during the medical procedure. FIG. 4A shows concertina distal ring 301 inserted through the colpotomy 403 in a closed configuration. Once inserted into the colpotomy 403, the concertina distal ring 301 may be actuated into an open configuration as shown in FIG. 4B where the diameter of concertina distal ring 301 is greater than the opening size of colpotomy 403. The opening of concertina distal ring 301 may allow for concertina ring medical device 300 to anchor to the body cavity 402 on the distal side of colpotomy 403.

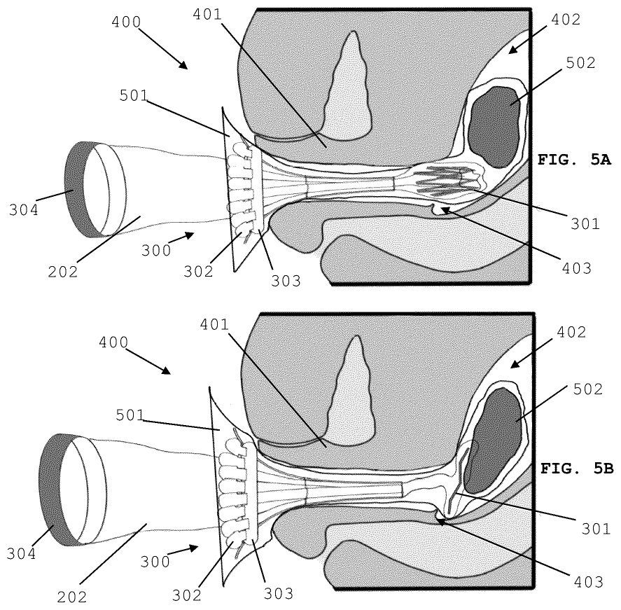

FIGS. 5A-5B shows concertina ring medical device 300 inserted into the inside of containment bag 501 situated in the body cavity 402 and vagina 400, according to an example. Containment bag 501 may be used for any medical procedure, e.g., a morcellation procedure on a tissue sample. The tissue containment bag 501 may be inserted in an opening (e.g., the vagina 400) prior to inserting a medical device (e.g., the concertina ring medical device 300). An opening of containment bag 501 may be extended out through colpotomy 403 and further through the vagina 400 to present its opening to the surgeon during a medical procedure. Containment bag 501 may contain a specimen 502 for removal, which could be any tissue specimen or organ to be removed from the body cavity 402 depending on the incision or natural orifice used to gain access. The insertion of concertina ring device 300 may provide the surgeon with access to specimen 502 in order to carry out necessary surgical techniques used to reduce its size and remove it, such as by cutting with scalpel through the retracted vaginal wall 401. In some instances sharp instruments may be inserted through the vagina 400, in order to reduce specimen 502 for removal. The guard petals 302 may protect the containment bag 501 and the vagina 400 from cutting or damage by sharp instruments during medical procedures.

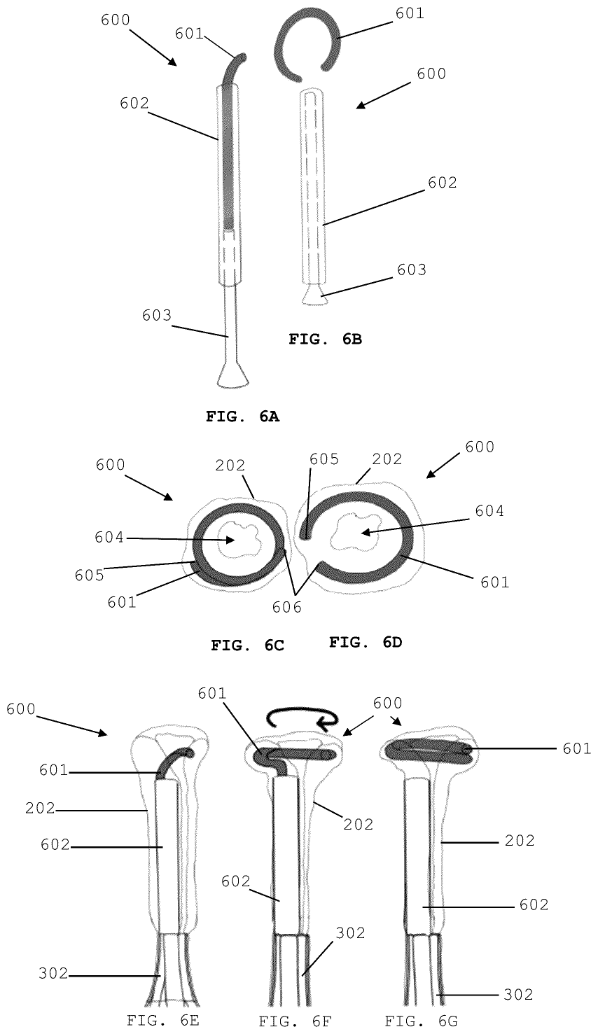

FIGS. 6A-6G show a show a severed ring medical device 600, according to an example, that includes a severed distal ring 601, an introducer tube 602 and an introducer plunger 603 for inserting a severed distal ring 601 into a body cavity. Such an introducer element severed ring device 600, may be used in conjunction with any medical device where a severed distal ring may be utilized to anchor the device in the body cavity through a body opening. Severed ring 601 may be constructed from a semi rigid plastic or rubber material. Severed distal ring 601 may have a pre-determined bias in shape towards a mostly circular configuration so that when ejected from introducer tube 602, it may serve as distal ring in a medical device. Inversely, the severed ring may have a pre-determined bias to be in a straight configuration.

FIG. 6A shows a severed ring 601 with a pre-determined circular bias, according to an example. As 601 is ejected from introducer tube 602 by depressing introducer plunger 603, the severed ring 601 may begin to return to its mostly circular configuration. FIG. 6B shows a fully ejected severed ring 601 having returned to its predetermined mostly circular shape when free from the confines of introducer tube 602. Severed ring 601 may be supplied in various lengths which could represent different overall equivalent distal ring sizes but by its nature, the severed ring 601 may vary in size when acted upon by an external force compressing the ring radially inward by increased the amount of overlap between the two free ends of severed ring 601 following ejection. In this way, the severed ring 601 may be variable to any increment between that of its largest and smallest sizes.

FIGS. 6C and 6D show examples of the variability of ring size while placing severed ring 601 within a sleeve 202. FIG. 6A shows a top-down section view of sleeve 202 when additional external force has been applied to sleeve 202, for example, in a small body cavity when the space in the body cavity does not allow for full expansion. While maintaining a sleeve central lumen 604, severed ring 601 increases the level of overlap between its free ends, free end 1 605 and free end 2 606, so that the ring effective size may be reduced to fit the space offered by the anatomy for example. FIG. 6D shows an example where the severed ring 601 is unrestricted in space and has no additional external forces acting upon it. Severed ring 601 may expanded to its pre-determined shape while maintaining sleeve central lumen 604.

FIGS. 6E-6G shows medical severed ring device 600 introducing severed ring 601 into the sleeve 202, which may receive an anchor distal ring, according to an example. The introducer tube 602 may be placed into an end (not depicted in FIGS. 6E-6C) of the sleeve 202 where the introducer plunger 603 may actuated the severed ring 601 to eject it into the sleeve 202. Severed ring 601 may begin to return to its pre-determined shape as it exits introducer tube 602. FIGS. 6F and 6G show the progression of the ejection of severed ring 601 where it enters the sleeve pocket of the medical device, decompresses, and acts as an anchor ring that may aid in securing the device in a body cavity.

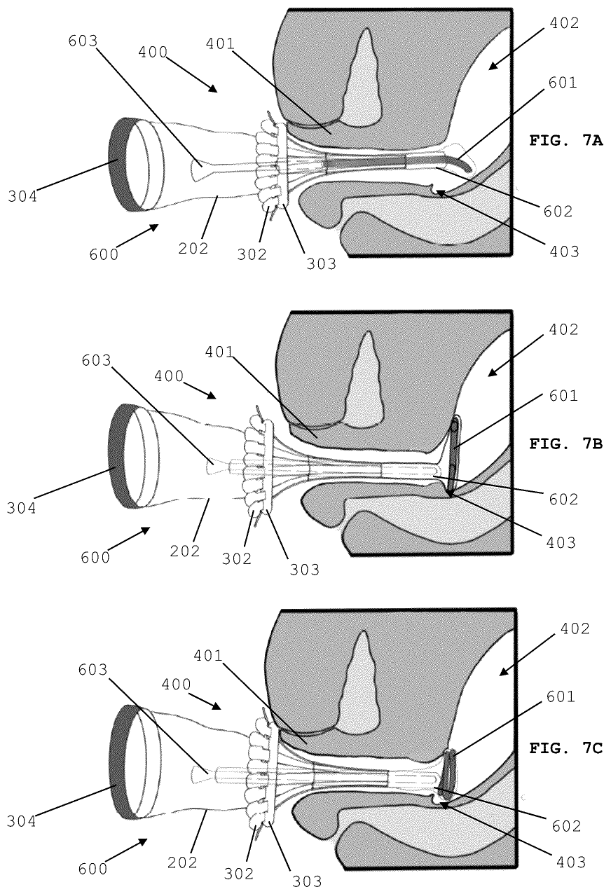

FIGS. 7A-7C illustrate that which is shown and described in FIGS. 6E-6G but within a vagina 400 and through colpotomy 403 into body cavity 402. Medical severed ring device 600 may be placed into the vagina 400. Introducer tube 602 may be inserted through the center of the medical severed ring device 600 and then through colpotomy 403 in order to reach body cavity 402. Once body cavity 402 is reached, the introducer plunger 603 may be actuated by an individual (e.g., surgeon) to eject severed ring 601 from the distal end of introducer tube 602 (FIG. 7A) into the pocket of sleeve 202. FIG. 7B shows an example where the severed ring 601 may be unrestricted by external forces and opens to its full predetermined shape in order to anchor distal to the colpotomy 403. FIG. 7C shows an example where the severed ring 601 may be compressed, for example, because of limited space in a small body cavity and may vary its size by increasing the overlap of its free ends free end 1 605 and free end 2 606, to create an anchor in a smaller body cavity.

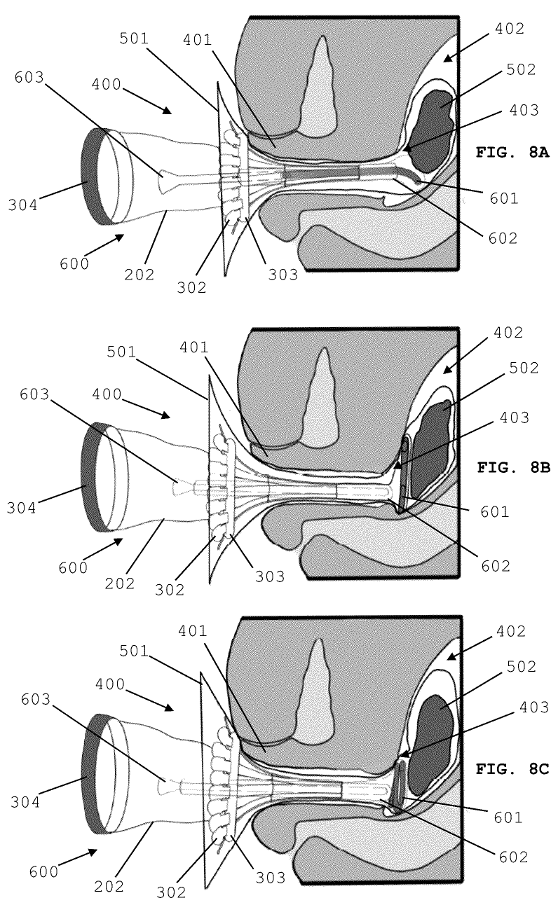

FIGS. 8A-8C repeats that which is described in FIGS. 7A-7C respectively but instead shows medical device severed ring device 600 inserted into the inside of containment bag 501 situated in the body cavity 402 and vagina 400, according to an example. The opening of containment bag 501 may be extended out through colpotomy 403 and further through the vagina 400 to present its opening to the surgeon during a medical procedure. Containment bag 501 contains a specimen 502 for removal which could be any tissue specimen or organ to be removed from the body cavity 402. The insertion of severed ring device 600 provides the surgeon with access to specimen 502 for removal in order to carry out necessary surgical techniques used to reduce its size and remove it, such as by cutting with scalpel through the retracted vaginal wall 401. In instances where sharp instruments may be used through the vagina 400 in order to reduce specimen 502 for removal for removal, guard petals 302 may protect the containment bag 501 and the vagina 400 from cutting or damage by sharp instruments.

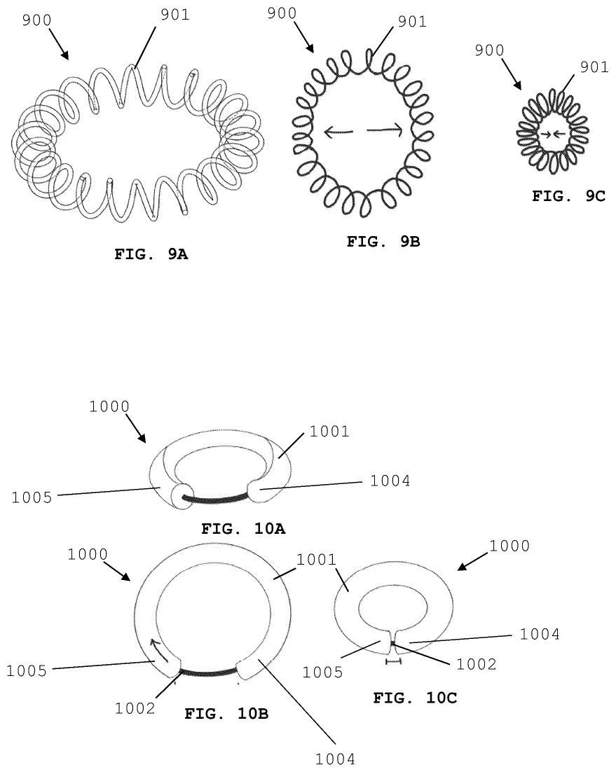

FIGS. 9A-9C shows a spring distal ring 900, according to an example, that may be used in any sleeved retractor medical device where a distal ring may be used to anchor the device in body cavity through a body opening. The spring distal ring 900 may be made from a metal or plastic where the properties may be such that the spring bounces back to its original size when all external forces are removed. The spring distal ring 900 may be made up of a series or spring turns 901 configured into a mostly circular shape as shown in FIG. 9A.

FIGS. 9B and 9C shows a top down view of spring distal ring 900, according to an example, in an open, uncompressed configuration (FIG. 9B) and in a compressed, small configuration (FIG. 9C). When external radially inward force is applied to spring distal ring 900 it may compress inwards (as depicted in the arrows of FIG. 8C), bringing spring turns 901 closer together to reduce the overall size of the circular shape. An example force may be that applied by the anatomy of a small body cavity. In this way, the spring distal ring 900 may be variable at any increment between that of its largest and smallest sizes.

FIGS. 10A-10C shows another ring embodiment, sliding ring device 1000, according to an example, as a possible component of a medical device including a solid outer ring element 1001 and a wire ring element 1002. The solid outer ring element 1001 may be constructed from a semi rigid plastic or rubber and wire ring element 1002 may be constructed from a more rigid wire or rigid plastic. The solid outer ring element 1001 may include a first end 1004 and a second end 1005, the two ends being at ends of an incomplete ring. The first end 1004 of the wire ring element 1002 may be fixed to the solid outer ring element 1001 and the second end 1005 of the wire ring element 1002 may be configured to slide along the wire ring element 1002. Through the application of external radially inward force, the wire ring element 1002 may be forced to slide into solid outer ring element 1001, thus effectively reducing the overall size of the ring that may be mostly circular. FIG. 10A shows the sliding ring device 1000 at its largest size and FIGS. 10B and 10C show sliding ring device 1000 reducing to and at its minimal size respectively. In this way, the sliding ring device 1000 may be variable at any increment between that of its largest and smallest sizes. In a similar way, the compression of the sliding ring device 1000 to its smallest size prior to insertion into the body cavity may improve the likelihood of the sliding ring device 1000 passing through the colpotomy into a body cavity without resistance and without causing harm to the patient.

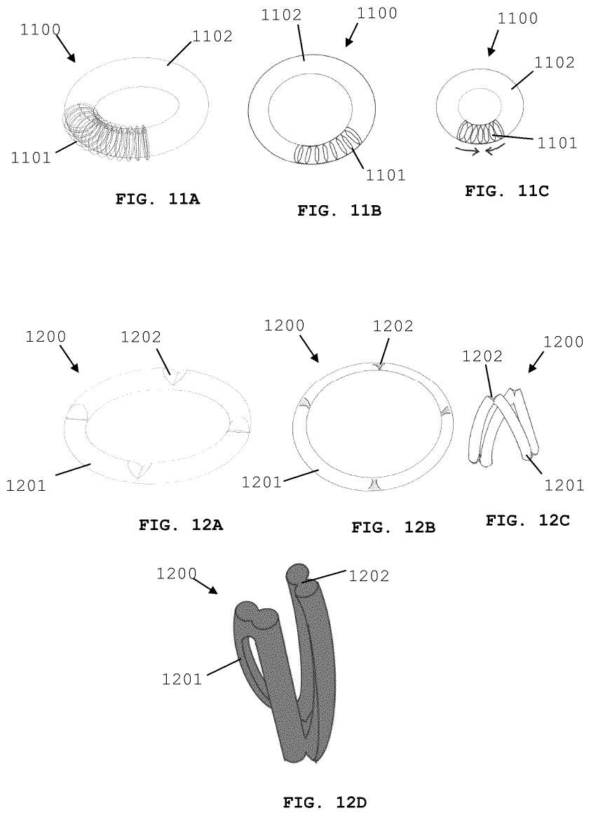

FIGS. 11A-11C shows another ring embodiment, a concertina section ring device 1100, according to an example, as a possible component of a medical device including a concertina ring section 1101 and a solid ring portion 1102. The concertina ring section 1101 and solid ring portion 1102 may be constructed from a semi rigid plastic or rubber. Concertina ring section 1101 may be fixed at both ends to solid ring portion 1102 and may be inherently compressible through its helix/spiral style construction. Through the application of external radially inward force, the concertina ring section 1101 may be forced to compress into itself, thus effectively reducing the overall size of the ring (e.g., reducing the diameter of the concertina section ring device 1100) that may be mostly circular. FIG. 11A shows the concertina section ring device 1100 at its largest size and FIGS. 10B and 10C show concertina section ring device 1100 reducing to and at its minimal size respectively. In this way, the concertina section ring device 1100 may be variable at any increment between that of its largest and smallest sizes. In a similar way, the compression of the concertina section ring device 1100 to its smallest size prior to insertion into the body cavity may improve the likelihood of the concertina section ring device 1100 passing through the to a body cavity without resistance and without causing harm to the patient.

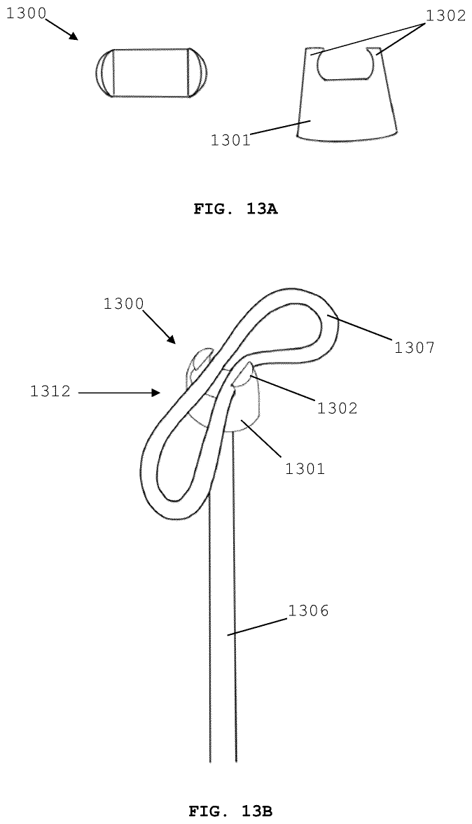

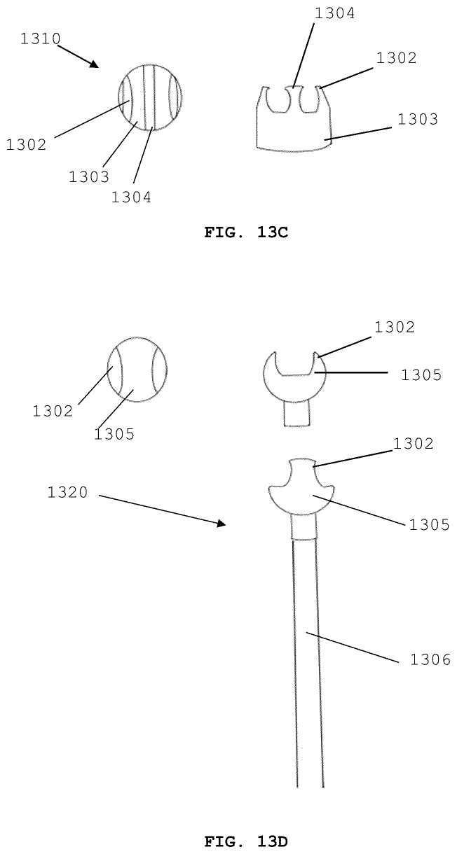

FIGS. 12A-12D shows another ring embodiment, divoted ring device 1200, according to an example. The devoted ring device 1200 may include a ring portion 1201 and one or more divots 1202. The divots 1202 may for example be hinge points. Divoted ring device 1200 may be constructed from a semi rigid plastic or rubber that may be flexible enough to bend when force may be applied but stiff enough to retain its circular shape in an unstressed state. Within the construct of divoted ring device 1200, there may be divots 1202 positioned at specific points in the ring's cross section that provide a focal point at which the devoted ring device 1200 may bend predictably. Divoted ring device 1200 may have any number of divotes 1202, for example there may be two, three, four, or five divots 1202 and so on to have the desirable collapsing effect. The one or more divots 1202 may be placed on difference planes within the cross section of the divoted ring device 1200, for example at 90, 180, 270 degrees as displayed in FIG. 12A in order to achieve the desired collapsing effect. FIGS. 12C and 12D show the perspective view of the divoted ring device 1200 in a collapsed configuration where a force has been applied and the divoted ring device 1200 bends at the desired divots in its cross section to collapse the divoted ring device 1200 effectively. The collapsing of the divoted ring device 1200 to its smallest size prior to insertion into the body cavity may improve the likelihood of the divoted ring device 1200 passing through the to a body cavity without resistance and without causing harm to the patient.

FIGS. 13A-13D shows a selection of pronged medical device anchoring ring introducers, according to an example. The pronged introducer device 1300, pronged introducer device 2 1310, and pronged introducer device 3 1320 that may allow for a medical device like many embodiments in this application to be inserted into a body cavity using a push rod style introduction. For example, the pronged introducer device 1300, pronged introducer device 2 1310, and pronged introducer device 3 1320 may all be configured to receive a portion of the collapsible rings described herein. This may include all of the various collapsible rings described in this disclosure.

FIG. 13A shows the pronged introducer device 1300, according to an example. The prong introducer device 1300 may include a tapered introducer head 1301 at a distal end 1312 of the shaft 1306 of the pronged introducer device 1300. The tapered introducer head 1301 may include a plurality of distally-extending prongs. The tapered introducer head 1301 may form a fork shape. The pronged introducer device 1300 may consist essentially of the shaft 1306 and the fork-shaped introducer head 1301. For example, the tapered introducer head 1301 may include two outer prongs 1302. The two outer prongs 1302 may be at a most distal end of the tapered introducer head 1301. Each of the two outer prong 1302 may be at an opposite side of the distal end of the tapered introducer head 1301. The tapered introducer head 1301 and outer prong 1302 may be made from a rigid or semi rigid plastic or rubber. As shown in FIG. 13B, the two outer prongs 1302 may retain a compressed standard ring 1307 which could also be any other style distal ring described in this application. Since the natural outer shape of the standard ring 1307 may be circular, there may be an inherent force acting outwards when the ring may be in a compressed state. This outward force may out against the outer prongs 1302 and in doing so, may engage with the outer prongs 1302 in such a way to retain the ring in the tapered introducer head 1301. The outer prongs 1302 may include a (e.g., two) curved/concaved inner face configured to receive the outer ring 1307. The curved/concaved inner face may for example match a curvature of an outer surface of the standard ring 1307 (or any of the distal anchoring member described in this application). The outer prongs 1302 curved faces may each face one another. In this way, the pronged introducer device 1300 may allow for a surgeon to insert a standard ring 1307 further into a body cavity where the body opening, for example a vagina, may be longer than the surgeon's reach by hand. The tapered sides of tapered introducer head 1301 may allow for easier insertion of pronged introducer device 1300 through an incision for example a colpotomy.

FIG. 13C shows a top and side view of another device introducer pronged introducer device 2 1310, according to an example, which includes two outer prongs 1302, a centre prong 1304 and a straight introducer head 1303. The centre prong 1304 may create a more secure grip on the ring when inserted between the two outer prongs 1302 in a compressed state. The straight introducer heat 1303 may be connected toa distal end of a shaft (e.g., shaft 1306 now shown).

FIG. 13D shows a top and side view of another device introducer pronged introducer device 3 1320, according to an example, which includes two outer prongs 1302, a rounded introducer head 1305 and an introducer shaft 1306. The rounded introducer head 1305 may allow for less resistance upon removal from a body cavity following insertion. The introducer shaft 1306 may be of varying lengths appropriate for insertion into incisions or natural orifices during surgery. For example, the introducer shaft 1306 may extend from a proximal end (not shown) to a distal end 1312. The length may be between 5 cm to 30 cm. In an example, when the introducer shaft 1306 is being utilized in a vagina, it may preferably be 5 cm to 20 cm. In an example, when the introducer shaft 1306 is being utilized in a vagina, it may preferably be 5 cm to 20 cm. In an example, when the introducer shaft 1306 is being utilized in a throat, it may preferably be 20 cm to 30 cm. In an example, when the introducer shaft 1306 is being utilized in an anal cavity, it may preferably be 5 cm plus or minus 2 cm.

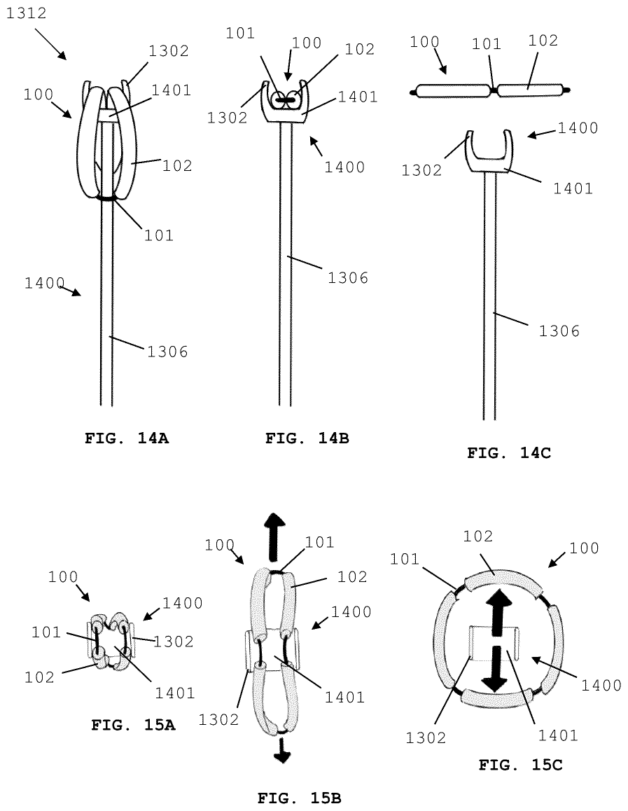

FIGS. 14A-14C shows a device introducer similar to that shown in FIGS. 13A-13D, where an introducer head pronged introducer head 4 1401 with two outer prongs 1302 sits at a distal end 1312 an introducer shaft 1306, according to an example. FIGS. 14A-14C shows pronged introducer device 4 1400 engaging with the collapsible wire ring 100 by way of demonstrating the insertion dynamics.

FIG. 14A shows the collapsible wire ring 100, according to an example, in a compressed state captured between two outer prongs 1302. Outer prong 1302 may retain the collapsible wire ring 100 and allow for the surgeon to further bend the collapsible wire ring 100 proximally to be adjacent with introducer shaft 1306 for insertion. In this, the footprint of the insertion device pronged introducer device 4 1400 in combination with the collapsible wire ring 100 may be minimised for insertion through an incision or natural orifice automatically. FIGS. 14B and 14C show the collapsible wire ring 100 being released partially and then totally from pronged introducer device 4 1400 respectively.

FIGS. 15A-15C represent FIGS. 14A-14C respectively from a top down view for illustrative purposes. The overall small minimal footprint of the introduction system pronged introducer device 4 1400 can be observed in FIG. 15A.

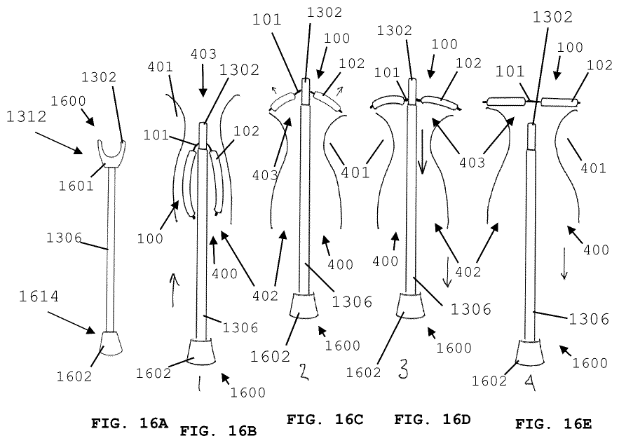

FIGS. 16A-16E expands further on this insertion example using another introducer device pronged introducer device 5 1600, according to an example, which includes an introducer head pronged introducer head 5 1601, two outer prongs 1302, an introducer shaft 1306 and an introducer shaft handle 1602. Introducer shaft handle 1602 may be located at a proximal end 1614 of the shaft 1306 and may be a feature of the pronged introducer device 5 1600 with which the surgeon may engage and handles during the insertion process.

FIG. 16B shows pronged introducer device 5 1600 in combination with the collapsible wire ring 100, according to an example, being inserted through a vagina 400 towards colpotomy 403 in order to access body cavity 402 (not shown). The slim profile of pronged introducer device 5 1600 in conjunction with the collapsible wire ring 100 may allow for the device to pass by the vaginal wall 401 with minimal resistance. Introducer shaft 1306 may allow for sufficient length (e.g., 5 cm to 30 cm) to reach all necessary anatomy with the device. The forces acting on the collapsible wire ring 100 by the vaginal wall 401 keep the ring to a slim profile throughout insertion. This may be made more possible by the collapsible nature of the collapsible wire ring 100 as described previously. FIG. 16C shows the collapsible wire ring 100 having been passed through the colpotomy 403 using pronged introducer device 5 1600. Once the collapsible wire ring 100 has passed through the colpotomy 403, the force from the vaginal wall 401 may be no longer acting on the collapsible wire ring 100 so the collapsible wire ring 100 may be allowed to spring back open symmetrically to either side. Upon subsequent retreat of pronged introducer device 5 1600, the now semi-open collapsible wire ring 100 may be pulled back against the distal side of the colpotomy 403 which may provide the force necessary to dislodge the collapsible wire ring 100 from outer prong 1302 to open completely, as shown in FIG. 16E. In this way, the collapsible wire ring 100 may be inserted, in conjunction with any medical device incorporating a distal anchor ring, into a body cavity that would otherwise be difficult to reach through hand insertion alone.

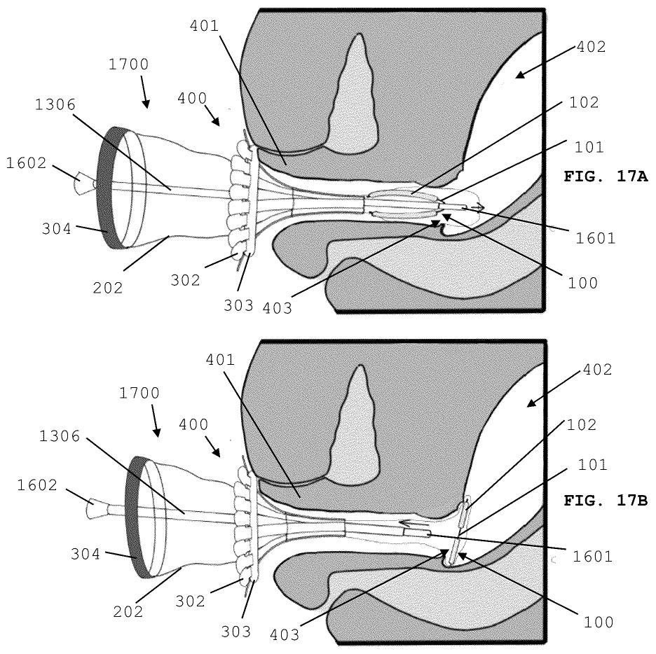

This insertion is further demonstrated in anatomical views in FIGS. 17A and 17B, according to an example, in conjunction with a sleeved medical device pre loaded ring retractor device 1700 where the collapsible wire ring 100 may be pre-loaded into the sleeve 202 for interaction with pronged introducer head 5 1601 during insertion.

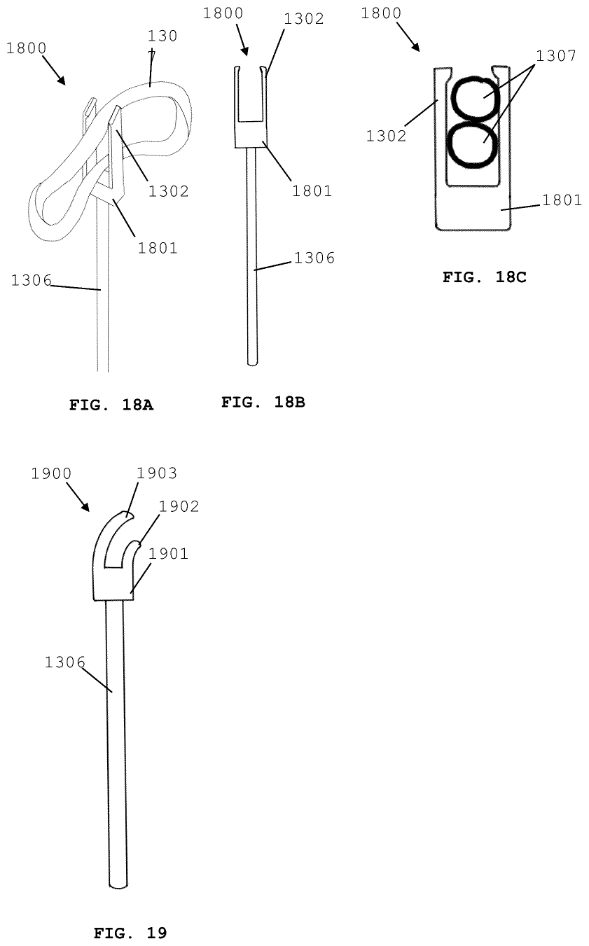

FIGS. 18A-18C show another introducer embodiment long introducer head device 1800, according to an example, that includes two outer prongs 1302, a long style introducer head 1801 and an introducer shaft 1306. The length of the two outer prongs 1302 may for example extend over 50% of the length of the long style introducer head 1801. In another example, the length of the two outer prongs 1302 may extend over 70% to 90% of the length of the long style introducer head 1801. In this embodiment, standard ring 1307 sits sideways between two outer prongs 1302 so that the two opposing cross sections of standard ring 1307 sit atop one another and the introducer head footprint may be reduced in width compared to earlier embodiments where the ring cross sections sit side by side in the introducer head. The cross-section view can be seen in FIG. 18C where the ring cross sections standard ring 1307 sit atop one another between the two outer prongs 1302.

FIG. 19 shows another introducer device with curved introducer head 1901, according to an example, an introducer shaft 1306 and two curved outer prongs short prong 1902 and long prong 1903. Short prong 1902 may be shorter than long curved prong 1903 but both prongs may curved to the same side as shown in FIG. 19. The curved nature of prongs short prong 1902 and long prong 1903 created a bias to one side during ring insertion when used in conjunction with a medical device that incorporates a distal anchor ring for insertion into a body cavity.

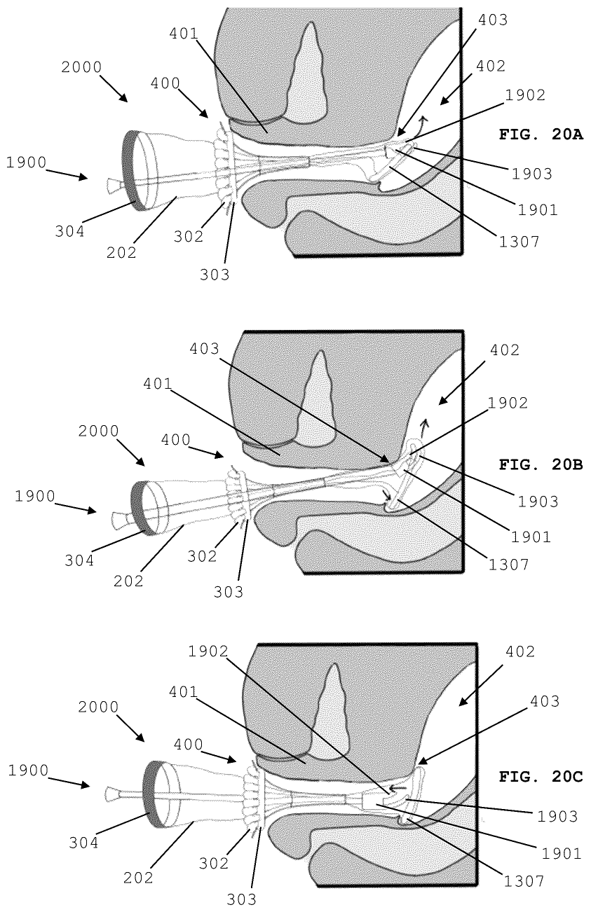

FIGS. 20A-20C illustrate the use of a medical device curved introducer retractor 2000, according to an example, that includes curved intro head device 1900. Standard ring 1307 may be hooked onto curved introducer head 1901 between curved prongs short prong 1902 and long prong 1903 in order to be pushed through the vagina 400 along with medical device curved introducer retractor 2000. The long prong 1903 may extend further and have a greater length than the short prong 1902. As indicated by the arrows in FIGS. 20A and 20B, the curved introducer head 1901 may bias the standard ring 1307 to one side during insertion so that the standard ring 1307 may be approximated closer to its desired final position. The bias may create a higher probability that the standard ring 1307 may be seated corrected in the body cavity 402 and less probability that it inadvertently dislodges when mounting the device past the colpotomy 403. FIG. 20C shows the proximal retreat and removal of curved intro head device 1900 following successful placement of the standard ring 1307 and device curved introducer retractor 2000 into the body cavity.

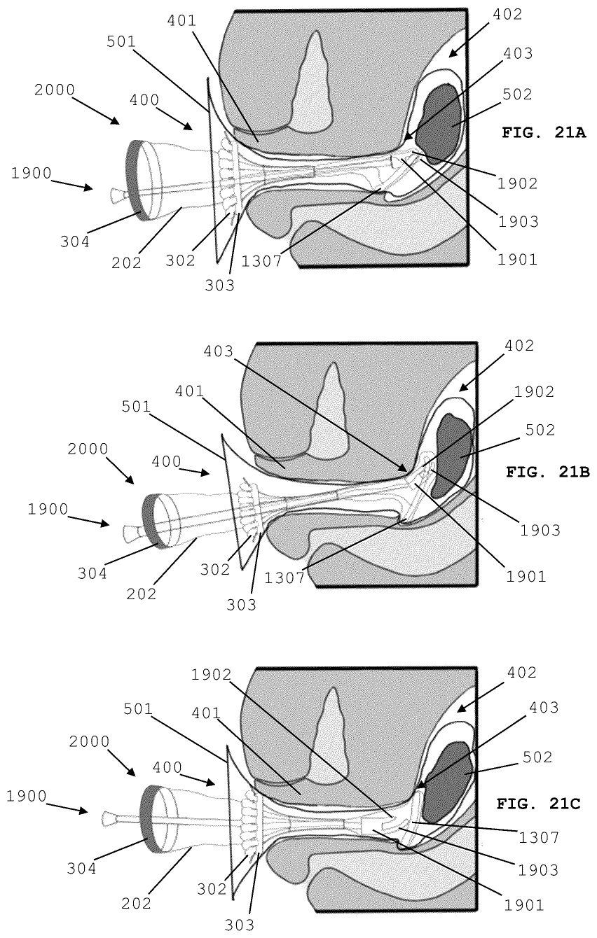

FIGS. 21A-21C show the same devices curved introducer retractor 2000 and curved intro head device 1900 demonstrated in anatomical view but with the addition of containment bag 501 and specimen 502 for removal, according to an example. The curved introducer head 1901 also may allow for the standard ring 1307 to be navigated away from specimen 502 for removal during insertion so as to increase the probability of a successful placement inside containment bag 501, inside body cavity 402.

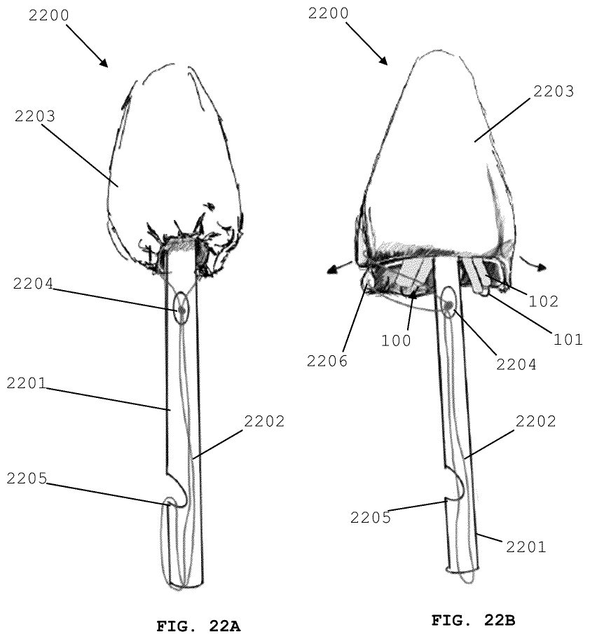

FIGS. 22A and 22B show a medical device hooded introducer device 2200, according to an example, for deployment of a ring, or distal ring incorporating device, into a body cavity. Medical device hooded introducer device 2200 may include a hooked introducer tube 2201, a drawstring 2202, a hood 2203, a drawstring hole 2204 and a drawstring hook 2205. Hooded introducer device 2200 may be used to insert a collapsible wire ring 100 shown in FIG. 22B. The hood 2203 may be at a distal end of the hooked introducer tube 2201 and may be a part of an introducer head. The hood 2203 may be configured to compress and maintain compression of a distal anchor ring (e.g., of the collapsible wire ring 100). Further, the hood may 2203 may reduce frictions between distal anchor ring and inner body walls (e.g., vaginal walls) during insertion of the medical device hooded introducer device 2200 into an opening of a patient. In medical device ring introducers previously mentioned in this application, it may have been beneficial to have an element that applies a compression force to the collapsible wire ring 100 in order to collapse it for insertion into a body cavity. Hooded introducer device 2200 includes a hood 2203 attached to the end of drawstring hook 2205. Drawstring 2202 may be fed through the drawstring hood channel 2206 circumferentially positioned at the open end of hood 2203. When the drawstring 2202 exits the drawstring hood channel 2206 it may then enter the drawstring hole 2204 in order to pass down through the centre of hooked introducer tube 2201. Upon exiting the bottom of hooked introducer tube 2201, drawstring 2202 may double back and may be stretched in tension onto drawstring hook 2205. In doing so, the drawstring 2202 may clinch closed drawstring hood channel 2206, and by extension, compresses and collapses the collapsible wire ring 100. In this configuration, hooded introducer device 2200 may be considered set in a locked configuration as shown in FIG. 22A. To release the ring from the introducer, a user may unhook drawstring 2202 from drawstring hook 2205 in order to release all tension in drawstring 2202 and uncinch drawstring hood channel 2206. By extension, this may allow for the hood 2203 to open under the opening spring force of the collapsible wire ring 100.

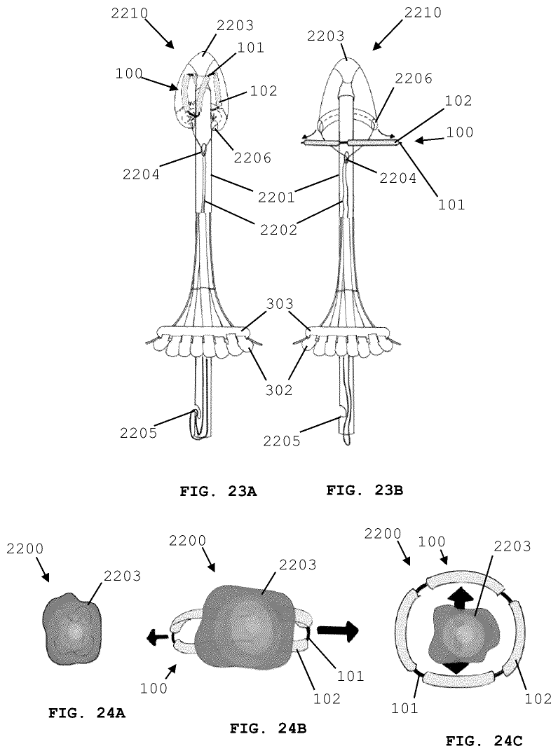

FIG. 23A and 23B show medical device hooded introducer device with guard 2210 integrated with a series of guard petals 302 and collapsible wire ring 100, according to an example. FIG. 23A shows the compressed configuration of the collapsible wire ring 100 through a transparent hood 2203.

FIGS. 24A-24C show the top view of hooded introducer device 2200 as the hood 2203 may be released in tension which may allow for the inherent spring force in the compressed collapsible wire ring 100 to push the hooked introducer tube 2201 away. In doing so, the collapsible wire ring 100 may be released and free to expand to its original circular uncompressed state as shown in FIG. 24C.

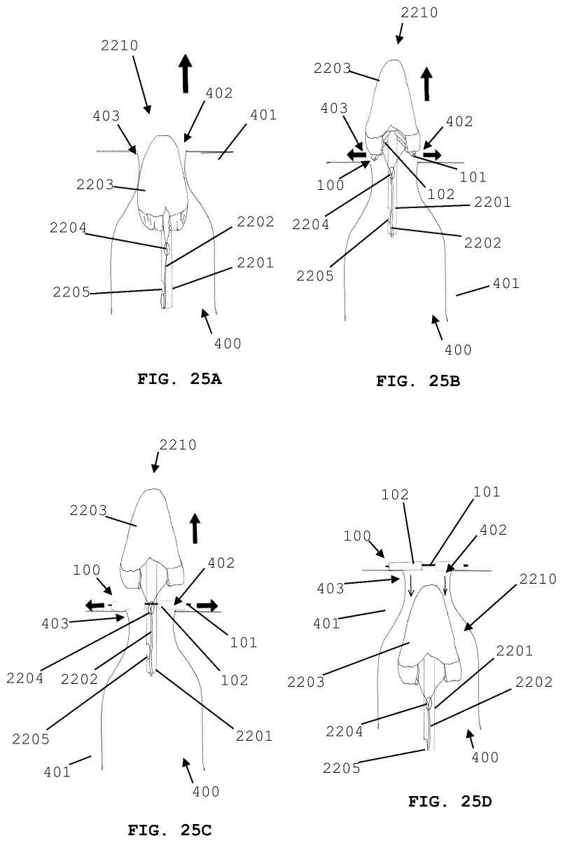

FIGS. 25A-25D further illustrate device hooded introducer device with guard 2210 in an anatomical view where it may be inserted into vagina 400 and through colpotomy 403 into body cavity 402. In FIG. 25B the hood may be passed through the colpotomy 403 to the body cavity 402. Upon reaching the body cavity the drawstring 2202 may be released to uncinch the hood from the collapsible wire ring 100. The collapsible wire ring 100 expands to its desired circular shape, forcing the hood 2203 to release the collapsible wire ring 100, FIG. 25C shows the collapsible wire ring 100 in a fully open configuration while the hood 2203 may be uncinched and disengaged entirely from the collapsible wire ring 100. In FIG. 25D the hooded introducer device with guard 2210 may be removed once more out of the vagina 400.

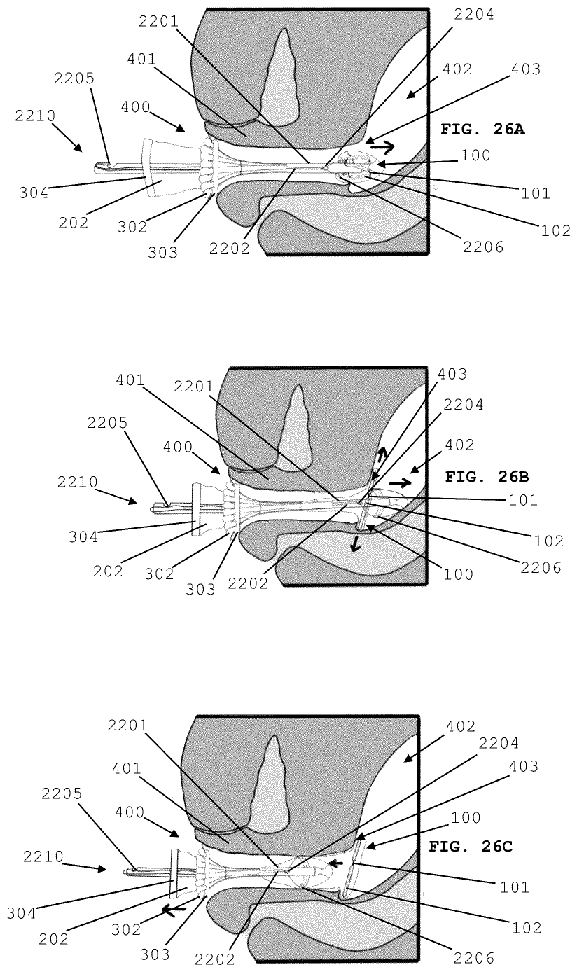

FIGS. 26A-26C further illustrate hooded introducer device with guard 2210 in another anatomical view. The inclusion of hooded introducer device with guard 2210 that compresses the collapsible wire ring 100 may remove the need for the surgeon to manually compress the collapsible wire ring 100 and also reduce the force acting outward from the collapsible wire ring 100 on the vaginal wall 401 and colpotomy 403 while inserting into the body cavity.

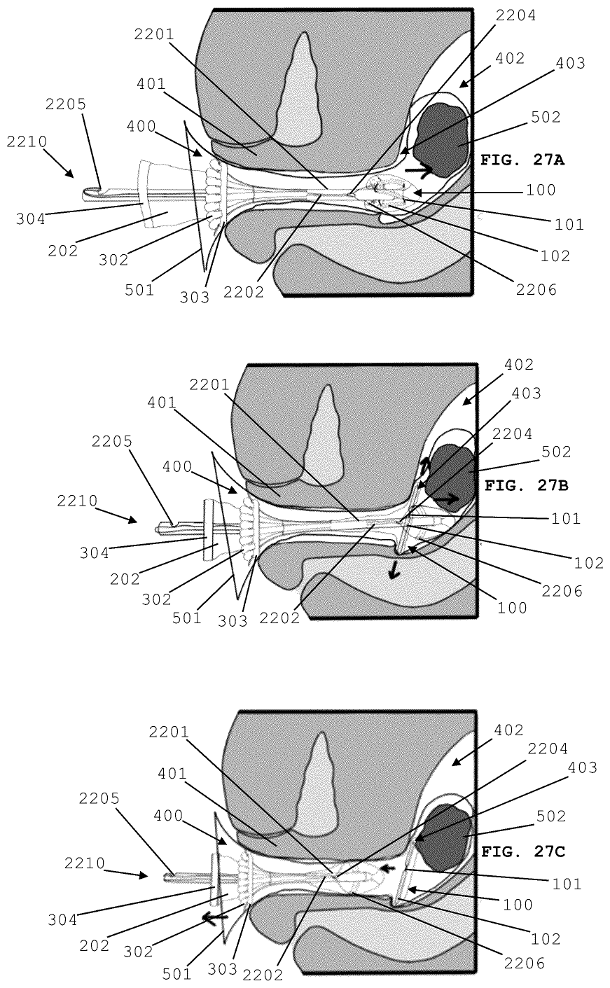

FIGS. 27A-27C further illustrate hooded introducer device with guard 2210 in the same anatomical view as FIGS. 26A-26C but with the inclusion of containment bag 501 and specimen 502 for removal, according to an example.

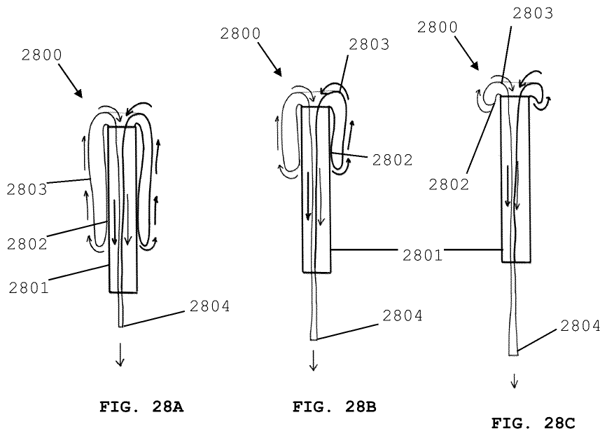

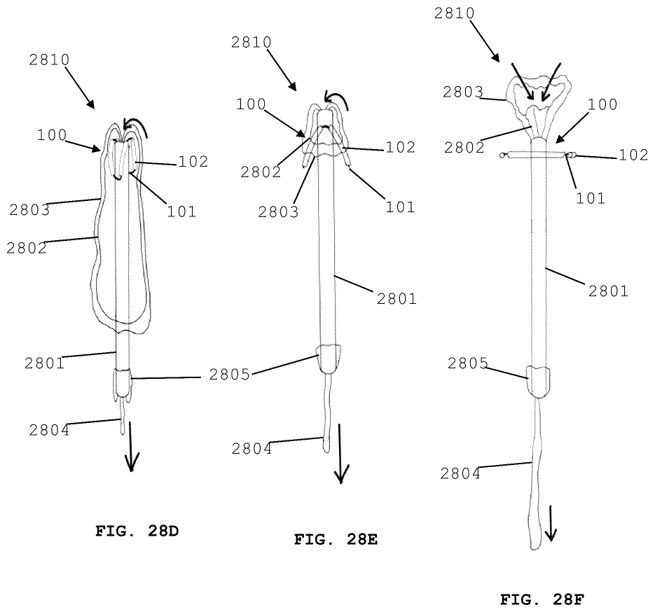

FIGS. 28A-28F show medical device sleeved introducer device 2800, according to an example, that includes a tube portion 2801, inner sleeve 2802, an outer sleeve 2803, and a sleeve free end 2804. The two layers of sleeve inner sleeve 2802 and outer sleeve 2803 may be passed through introducer tube portion 2801, and doubled back around the outside of tube portion 2801 to form a two layered wrapping tube around tube portion 2801. When the free end of sleeve free end 2804 is pulled proximally the outer sleeve moves relative to the inner sleeve 2802 in a rolling type fashion. As the outer sleeve may be moved around the outside of inner sleeve 2802, inner sleeve 2802 begins to peel away distally from the surface of the tube in a non-manner without any friction. Continuing to pull sleeve free end 2804, as shown in FIG. 28B may shorten the wrapping on the outside of tube to tube portion 2801 as the outer sleeve 2803 peels inner sleeve 2802 away from its surface with no friction. FIG. 28C shows the inner sleeve and outer sleeve almost entirely removed from the outside of tube portion 2801.

FIGS. 28D-28F show sleeved introducer device with ring 2810 that includes all the elements of sleeved introducer device 2800 in conjunction with the collapsible wire ring 100, according to an example. In FIG. 28D the free end of inner sleeve 2802 may be fixed in position at the bottom of tube portion 2801, this fixed portion of inner sleeve 2802 may be a sleeve fixation point 2805. By fixedly connecting inner sleeve 2802 to tube portion 2801, the sleeve may have means to an equal and opposite force when the sleeve free end 2804 of the sleeve may be pulled proximally. Within the wrapping tube created by doubling back both layers of sleeve inner sleeve 2802 and outer sleeve 2803, the collapsible wire ring 100 may be encapsulated and compressed for insertion. FIG. 28E shows the sleeve free end 2804 being pulled proximally. In doing so, the outer sleeve 2803 translates back down the tube portion 2801 relative to the inner sleeve 2802. For each increment of outer sleeve 2803 that pulls backward the same amount of inner sleeve 2802 that may be peeled up and away from the surface of tube portion 2801 in a non-friction manner, similar to the motion seen in a tank caterpillar track or belt. Continued pulling of sleeve free end 2804 may ultimately lead to the exposure of the collapsible wire ring 100, otherwise restricted in a compressed configuration within the doubled back sleeve tube. FIG. 28F shows that with continued pulling of sleeve free end 2804 downward the inner sleeve 2802 that may be completely peeled away and removed from the tube portion 2801 and may fully release the collapsible wire ring 100 to expand to its full circular configuration to present itself for anchoring in the body cavity.

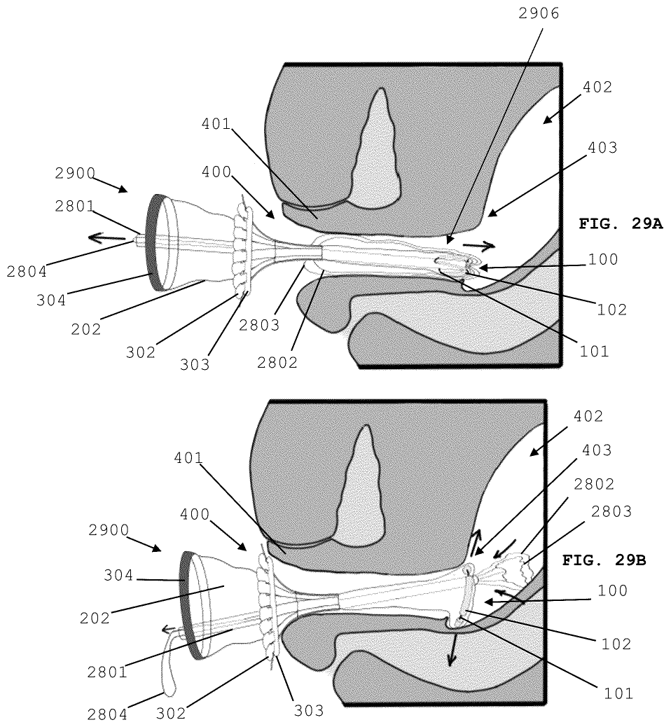

FIGS. 29A and 29B show evolution of sleeved introducer device with ring 2810 to incorporate with a guarded sleeve retractor to become medical device with sleeved introducer 2900, according to an example. In this embodiment, the collapsible wire ring 100 may be pre-loaded into the sleeve 202 at a distal end 2906 of the device to be loaded into the introducer element. The tube portion 2801 may be fed down through the centre of sleeve 202 as far as the collapsible wire ring 100 where the collapsible wire ring 100 may be compressed against tube portion 2801. The inner and outer sleeves inner sleeve 2802 and outer sleeve 2803 may be wrapped backwards around the already compressed collapsible wire ring 100 to retain in inside the doubled back tube wrapping in a compressed state. The medical device with sleeved introducer 2900 may be then inserted into the vagina 400 and passed through the colpotomy 403 into body cavity 402. Once collapsible wire ring 100 is positioned past colpotomy 403, the sleeve free end 2804 may be pulled proximally to initiate the peeling of inner sleeve 2802 away from the tube portion 2801 and collapsible wire ring 100. FIG. 29B shows the inner sleeve fully peeled away from tube portion 2801 and collapsible wire ring 100, leaving the collapsible wire ring 100 to spring open to its full circular configuration ready to anchor behind the colpotomy 403 inside the body cavity 402. Following the deployment of collapsible wire ring 100, the introducer elements tube portion 2801, inner sleeve 2802, outer sleeve 2803 and sleeve free end 2804 may be removed from the vagina 400.

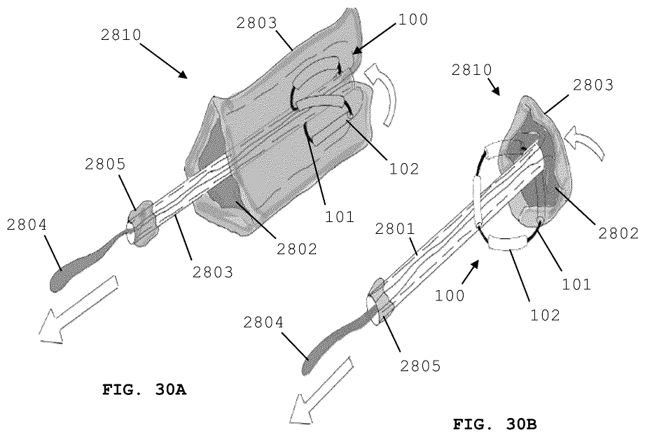

FIGS. 30A and 30B show perspective views of sleeved introducer device with ring 2810 and the progression from a compressed insertion view (FIG. 30A) to an uncompressed (FIG. 30B) view of deployed collapsible wire ring 100 where the sleeve elements inner sleeve 2802 and outer sleeve 2803 have been peeled back from tube portion 2801 and collapsible wire ring 100 by pulling proximally on the sleeve free end 2804.

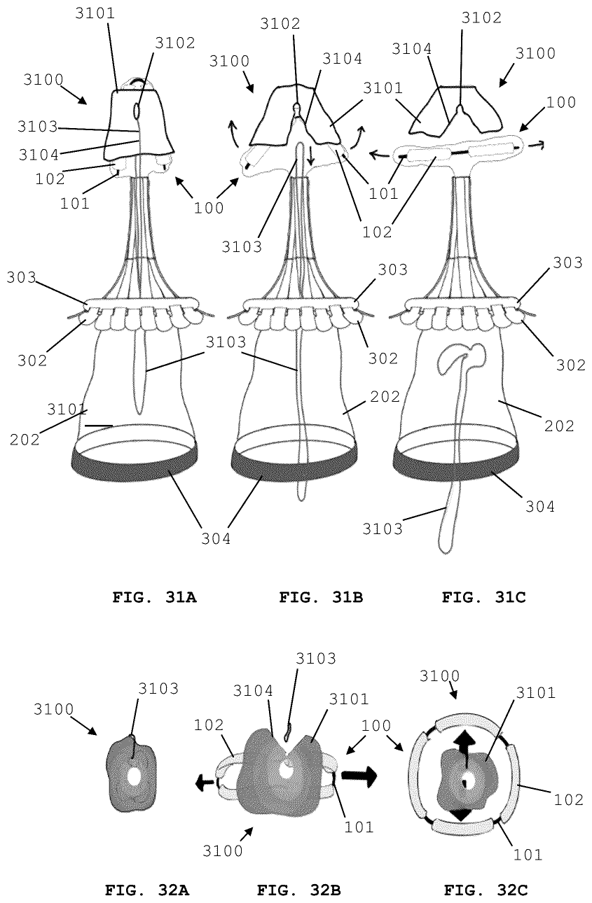

FIGS. 31A-31C show a medical device tear away introducer device 3100, according to an example, that includes a guard ring 303, guard petal 302, sleeve 202, proximal ring 304, a tear away hood 3101, a tear away starting hole 3102, a tear away drawstring 3103 and a hood weakened section 3104. The collapsible wire ring 100 may be preloaded into the sleeve 202 to prepare for insertion into a body cavity through an incision or natural orifice. Tear away hood 3101 may be placed over a compressed collapsible wire ring 100 in order to retain the collapsible wire ring 100 in a compressed configuration. Tear away hood 3101 has a tear away starting hole 3102 through which a tear away drawstring 3103 may be looped through in order to return tear away starting hole 3102 up through the device to the user as a free end. Through a technique similar to that of a cheese wire, the tear away starting hole 3102 may be pulled to initiate a cutting or tearing of the tear away hood 3101 along a possible hood weakened section 3104. In FIG. 31B the tear away hood 3101 has been torn by the pulling of the tear away drawstring 3103 by the surgeon. As the tear away hood 3101 now has a compromised structure, it may no longer retain the collapsible wire ring 100 in a compressed state owing to the inherent spring forces within a compressed collapsible wire ring 100. The outward acting force of the collapsible wire ring 100 may be enough to overcome the comprised structure of tear away hood 3101 and therefore, may be able to open fully as shown in FIG. 31C. The collapsible wire ring 100 may now act freely as the anchor ring for medical device tear away introducer device 3100 in the body cavity.

FIGS. 32A, 32B and 32C show the top down view of FIGS. 31A, 31B and 31C respectively, of tear away introducer device 3100.

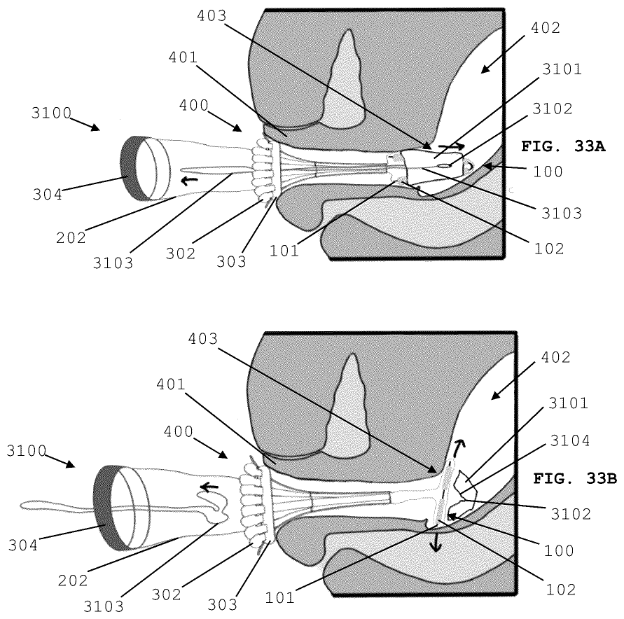

FIGS. 33A and 33B show tear away introducer device 3100 being inserted through a vagina 400 into a body cavity 402 in order to gain access to a body cavity for a surgical procedure. Once collapsible wire ring 100 and tear away hood 3101 are progressed past the colpotomy 403, the surgeon may pull on 3103 so that the tear away hood 3101 may be torn along its hood weakened section 3104. In FIG. 33B the tear away hood 3101 is torn and the collapsible wire ring 100 may be allowed to spring open to its original circular shape. The collapsible wire ring 100 may be now positioned in the body cavity 402 behind the colpotomy 403 in order to serve as an anchoring element for tear away introducer device 3100 in the body cavity 402.

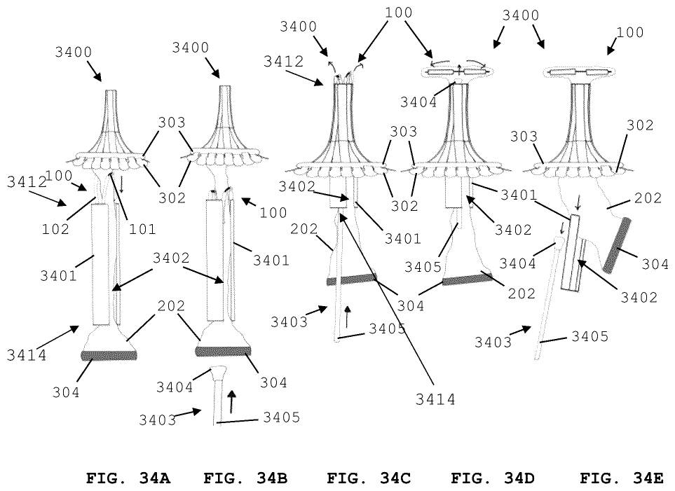

FIGS. 34A-34E illustrate a slotted tube introducer device 3400, according to an example, that may be used to facilitate insertion of a medical device into a body cavity, in particular, a medical device with an incorporated distal ring for anchoring said device in the body cavity. The slotted tube introducer device 3400 includes a slotted introducer tube 3401, a slot 3402 and a wide tip introducer plunger 3403 and is shown in FIG. 34A in conjunction with a sleeved medical device including collapsible wire ring 100, guard petal 302, guard ring 303, proximal ring 304 and sleeve 202. In FIGS. 34A and 34B the slotted introducer tube 3401 may be positioned with sleeve 202 up through its centre opening. The collapsible wire ring 100 may be fed inside the distal end 3412 of slotted introducer tube 3401 where it may be retained in a compressed state for insertion. In FIG. 34C the slotted introducer tube 3401 may be inserted through the centre of the guard petal 302 and guard ring 303 to emerge out of the distal end 3412 of guard petal 302. A plunger shaft 3405 may be used by inserting its wide tip introducer plunger 3403 into the proximal opening 3414 of slotted introducer tube 3401. A distal head 3404 of the plunger 3403 may be located at a distal end of the plunger 3403. The plunger shaft 3405 may be pushed until it reaches collapsible wire ring 100 and proceeds to push collapsible wire ring 100 out of the distal end of slotted introducer tube 3401 and out the distal end of the guard petal 302 as shown in FIG. 34D. FIG. 34E shows the removal of plunger shaft 3405 and slotted introducer tube 3401 following the deployment of collapsible wire ring 100. Plunger shaft 3405 and slotted introducer tube 3401 may be retreated out the proximal opening 3414 of the slotted tube introducer device 3400. In order to facilitate its separation from sleeve 202, the slotted introducer tube 3401 may have a slot 3402 along its full length (e.g., from proximal opening 3414 to distal end 3412). In that way, sleeve 202 may slot out through slot 3402 to free slotted introducer tube 3401 from the device following the deployment of collapsible wire ring 100. Slotted introducer tube 3401 may include a clip or fastener to minimise the diameter of slotted introducer tube 3401 for insertion and to minimise the width of slot 3402. The clip or fastener may be untied or released after deployment to facilitate separation of 3401 from the slotted tube introducer device 3400.

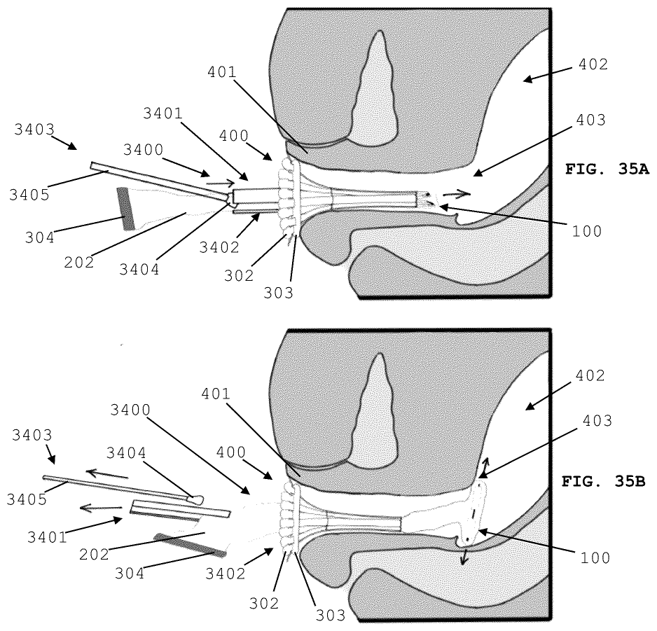

FIGS. 35A and 35B show slotted tube introducer device 3400 being utilised in an anatomical view where slotted tube introducer device 3400 may be inserted through a vagina 400 and colpotomy 403 in order to gain access into a body cavity 402 for a surgical procedure. The collapsible wire ring 100 may be preloaded into slotted introducer tube 3401 and progressed down the centre of slotted tube introducer device 3400 until reaching past the colpotomy 403, at which point plunger shaft 3405 may be inserted to push collapsible wire ring 100 out the distal end of slotted introducer tube 3401 to release it into the sleeve 202 and into the body cavity 402. Both wide tip introducer plunger 3403 and slotted introducer tube 3401 may be removed following the deployment of collapsible wire ring 100 and this may be facilitated by slot 3402. Collapsible wire ring 100 may be left in the sleeve 202 within the body cavity 402 to serve as the anchoring element or the medical device slotted tube introducer device 3400 throughout the medical procedure.

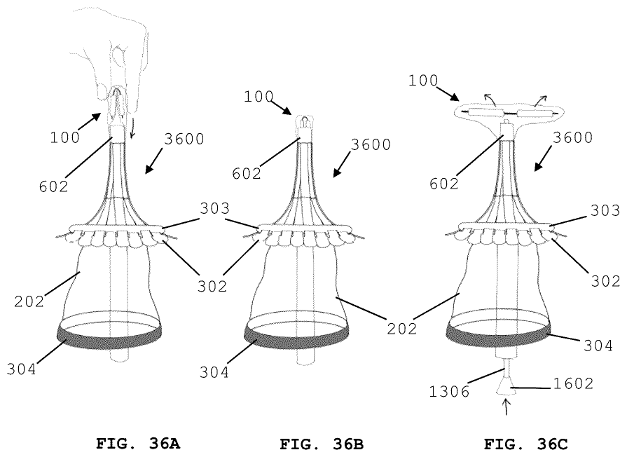

FIGS. 36A-36C shows a medical device complete with tubing introducer device 3600, according to an example, that may be very similar to slotted tube introducer device 3400. The device includes instead an introducer tube 602 without any slot 3402 seen in slotted tube introducer device 3400. In this instance, the introducer tube 602 may only be inserted inside sleeve 202 in order to be removable from the device after deploying the collapsible wire ring 100. Collapsible wire ring 100 may be compressed to a collapsed state and fed inside the introducer tube 602 distal end as shown in FIG. 36B. Introducer shaft 1306 may then be pushed down introducer tube 602 using introducer shaft handle 1602 in order to eject collapsible wire ring 100 from the distal end of introducer tube 602. Introducer shaft 1306, introducer shaft handle 1602 and introducer tube 602 may then be removed from the tubing introducer device 3600 leaving collapsible wire ring 100 left in the sleeve 202 to serve as the anchoring element or the medical device tubing introducer device 3600 throughout the medical procedure.

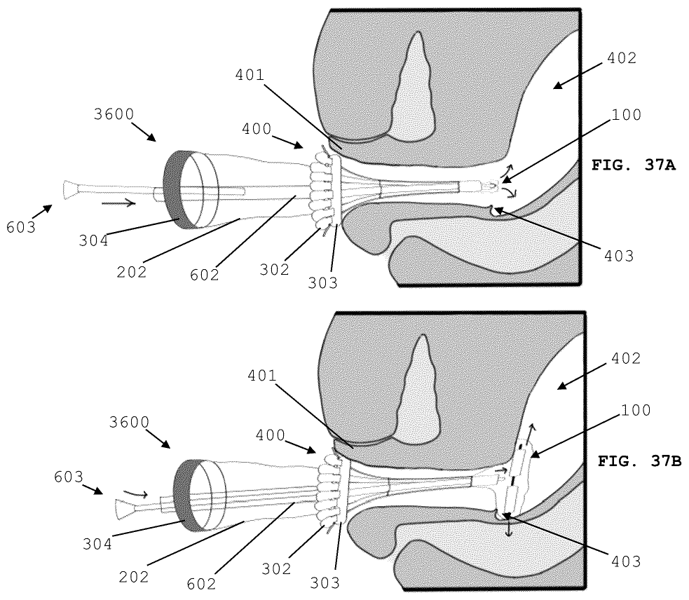

FIGS. 37A and 37B show tubing introducer device 3600 being utilised in an anatomical view where tubing introducer device 3600 may be inserted through a vagina 400 and colpotomy 403 in order to gain access into a body cavity 402 for a surgical procedure. The introducer tube 602 may be progressed down the centre lumen of tubing introducer device 3600, where collapsible wire ring 100 may be loaded into distal end of introducer tube 602. Tubing introducer device 3600 may be progressed until reaching past the colpotomy 403, at which point introducer plunger 603 may be inserted to push collapsible wire ring 100 out the distal end of introducer tube 602 to release it into the sleeve 202 and into the body cavity 402. Both introducer plunger 603 and introducer tube 602 may be removed following the deployment of collapsible wire ring 100. Collapsible wire ring 100 may be left in the sleeve 202 within the body cavity 402 to serve as the anchoring element or the medical device tubing introducer device 3600 throughout the medical procedure.

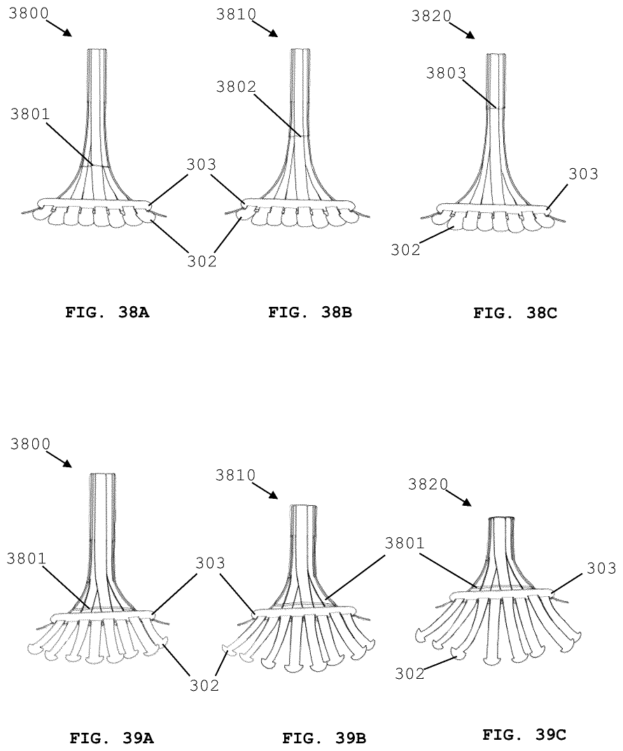

FIGS. 38A-38C show, in isolation, guard elements long petal skirt guard 3800 (FIG. 38A), medium petal skirt guard 3810 (FIG. 38B) and short petal skirt guard 3820 (FIG. 38C) of a medical device, according to an example. The three different petal skirts of FIG. 38A-38C may have varying length. Generally, the long petal skirt 3801, medium petal skirt 3802 and short petal skirt 3803 of the guard may be a section of film or similar material of a defined length that captures the guard petals 302 by way of welding or other form of fixation in order to give the petals an overall organization and structure when handled and retracted in use. Long petal skirt 3801, medium petal skirt 3802 or short petal skirt 3803 also may serve as a stopping point for the guard ring 303 when the guard ring 303 slides down the guard petals 302 during use. In use, the guard ring 303 may be forced distally relative to the guard petals 302 when pushed by the surgeon either directly or indirectly through the actuation of a retractor sleeve 202 shown in previous complete embodiments. The guard petals 302 may have a preferred length between length 50-200 mm. The short petal skirt 3803 may have a length of 1-30% of overall petal length of the guard petals 302, the medium petal skirt 3802 may have a length of 30-70% of overall petal length of the guard petals 302, and the long petal skirt 3801 may have a length of 70-100% of overall petal length of the guard petals 302.

FIGS. 39A-39C show this motion for devices long petal skirt guard 3800, medium petal skirt guard medium petal skirt guard 3810 and short petal skirt guard 3820 respectively. Guard ring 303 may move freely down the guard petals 302 via holes or slots in its construction but may come to a stop (e.g., a stop point) when the ring meets the top (e.g., proximal end) of the petal skirts long petal skirt 3801, medium petal skirt 3802 and short petal skirt 3803. In this way, the length of the petal skirt may be defined in such a way to limit the allowable movement of the guard ring 303 and, by extension, limit the reach of the guard petals 302 into the body opening or incision during surgery. In serving as a stop point the petal skirts may dictates the minimum distance the petals 302 (and therefore the protection they offer) extend into the incision/natural orifice when the device is actuated (e.g., any of the medical devices described in the application). The long petal skirt 3801, medium petal skirt 3802 or short petal skirt 3803 may give structure and organization to the distal end of the petals during insertion and retraction.

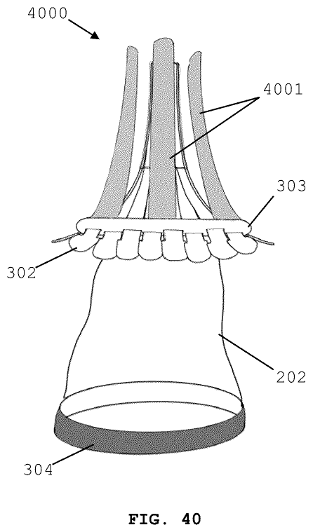

FIG. 40 shows medical device outer sheath device 4000, according to an example. The medical device outer sheath device 4000 may includes a proximal ring 304, sleeve 202, guard petal 302, guard ring 303 and introducer tube with funnel 4001. Outer sheaths introducer tube with funnel 4001 may be affixed to the guard ring 303 to the outside of the guard petals 302. During insertion of outer sheath device 4000, guard petals 302 first, into an incision or body opening, the sheaths introducer tube with funnel 4001 shield the guard petals 302 from the friction associated with the insertion. If a surgeon holds the guard ring 303 during insertion and friction acts upon the guard petals 302 during this insertion of outer sheath device 4000 into, for example, a vagina then guard petals 302 may prematurely extend out through the guard ring 303 proximally, effectively shortening the reach of the guard petals 302 and/or the outer sheath device 4000 as a whole into the patient. The outer sheaths introducer tube with funnel 4001 may shield the guard petals 302 from the friction associated with insertion and prevent the premature shortening of the effective length of the guard petals 302 and/or outer sheath device 4000.

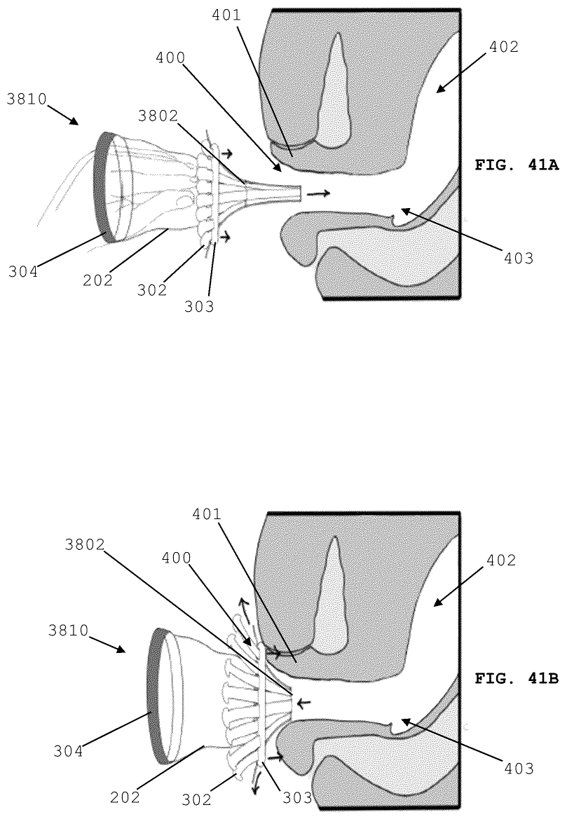

FIGS. 41A and 41B show the effective shortening of guard petal 302 reach in the anatomy while using medium petal skirt guard 3810 in conjunction with sleeve 202 and proximal ring 304. The surgeon's hand may be pushing the guard ring 303 distally and when the guard petals 302 meet resistance and friction in the vagina 400 while contacting the vaginal walls 401 (FIG. 41B) the guard petals 302 extend outward through the guard ring 303 instead of progressing further into the vagina 400. These FIGS. 41A and 41B illustrate the impact that premature petal extension may have of the effective length of the medium petal skirt guard 3810.

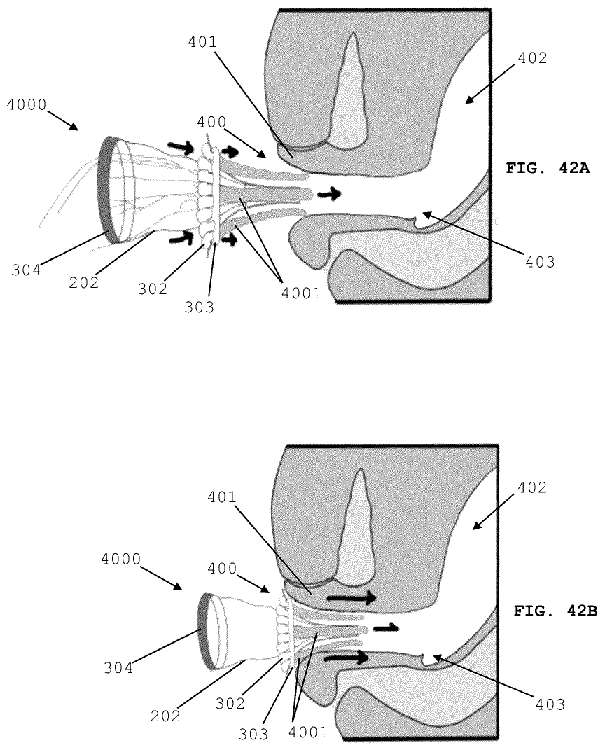

By comparison, FIGS. 42A and 42B show outer sheath device 4000 complete with introducer tube with funnel 4001 being inserted into a vagina 400 by pushing distally on the guard ring 303 in the same way shown in FIGS. 41A and 41B, according to an example. The sheaths introducer tube with funnel 4001 shield the guard petals 302 from the friction exerted by the vaginal walls 401 during insertion, thus potentially allowing for the petals to progress to the full allowable length into the vagina 400. No premature extension of the guard petals 302 has occurred. Introducer tube with funnel 4001 may exist as numerous individual pieces or could be one continuous sheath on the outside of the petals and be made from any flexible or semi rigid or rigid material.

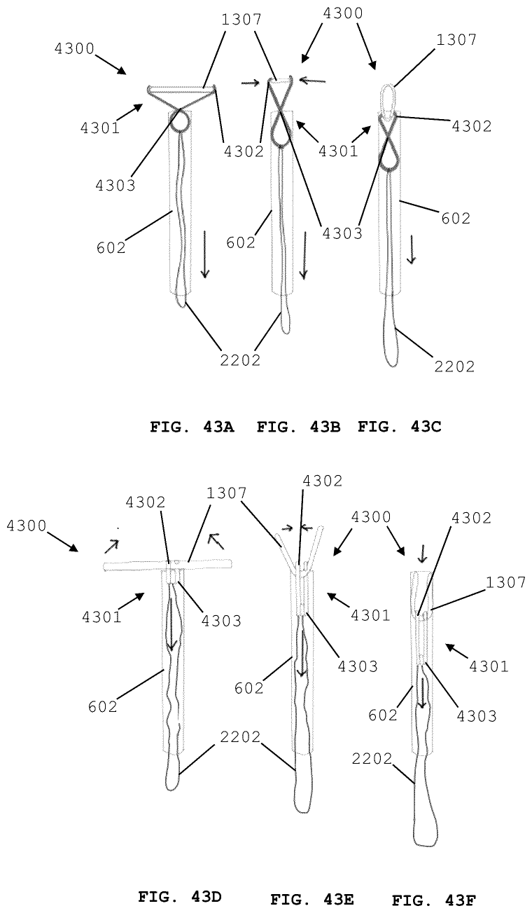

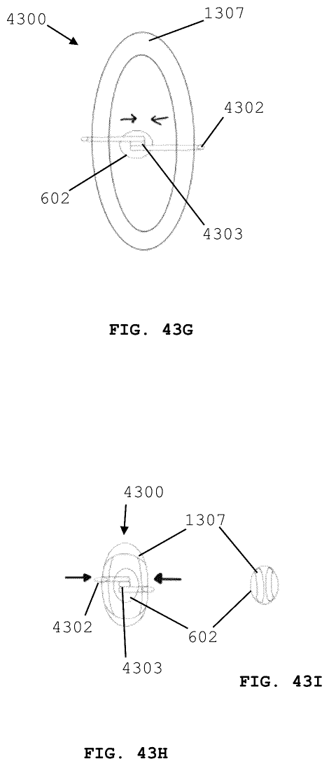

FIGS. 43A-43I show a distal standard ring 1307 sprung clasp introducer device 4300, according to an example. The sprung clasp introducer device 4300 may include an introducer tube 602, a sprung ring holder 4301, a sprung arm 4302, an arm crossover section 4303 and drawstring 2202. FIGS. 43A-43C show front view, FIGS. 43D-43F show side view and FIGS. 43G-43I show top view of similar steps in the device's operation respectively. The sprung ring holder 4301 may be made from a sprung metal or plastic material that offers some flexibility in the part while possessing inherent spring force to bias the part back to its open configuration when compressed. The sprung ring holder 4301 may include two sprung arms 4302 and an arm crossover section 4303. The two sprung arms 4302 may cross each other at arm crossover section 4303, creating a mechanism akin to a partial turn of a spring with a spring diameter sized to fit inside the opening of introducer tube 602. When the sprung arms 4302 are protruding out of the end of introducer tube 602, they may be unrestricted and open to their full or near full extent as shown in FIGS. 43A, 43D and 43G. In this position, the standard ring 1307 may be placed in between the two sprung arms 4302 as a holder of the ring in position. When drawstring 2202, which may be attached to sprung ring holder 4301, may be pulled downwards the sprung arms 4302 may be forced inward towards a closed position by the walls of the introducer tube 602. As the sprung arms 4302 close, they may compress the standard ring 1307 at the same time as dragging it backward into the tube (FIGS. 43B, 43E and 43H) until such a time that it may be completely contained within the introducer tube 602 (FIGS. 43F and 43I).

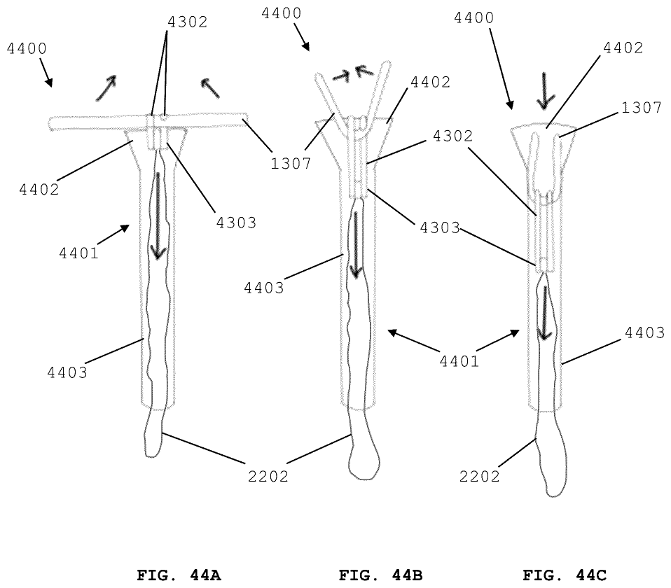

FIGS. 44A-44C show a medical device sprung ring holder funnelled tube 4400, according to an example. The medical device sprung ring holder funnelled tube 4400 may be almost identical to sprung clasp introducer device 4300 but instead, the introducer tube with funnel 4401 may have both a straight section tube portion 4403 and flared funnelled opening 4402 at its distal end where arm crossover section 4303 and funnelled opening 4402 compress and drag standard ring 1307 into the introducer tube with funnel 4401 in much the same way as shown in FIGS. 43A-43I. The flared funnelled opening 4402 may facilitate an easier transition of the standard ring 1307 into the introducer tube with funnel 4401 by angling the opening like a funnel.

In such an embodiments, sprung clasp introducer device 4300 and sprung ring holder funnelled tube 4400, in the absence of a plunger or rod to then eject the standard ring 1307 by pushing arm crossover section 4303 distally again and into an open configuration, it may instead be ejected using any medical instrument long enough that may be commonly used in laparoscopic surgery.

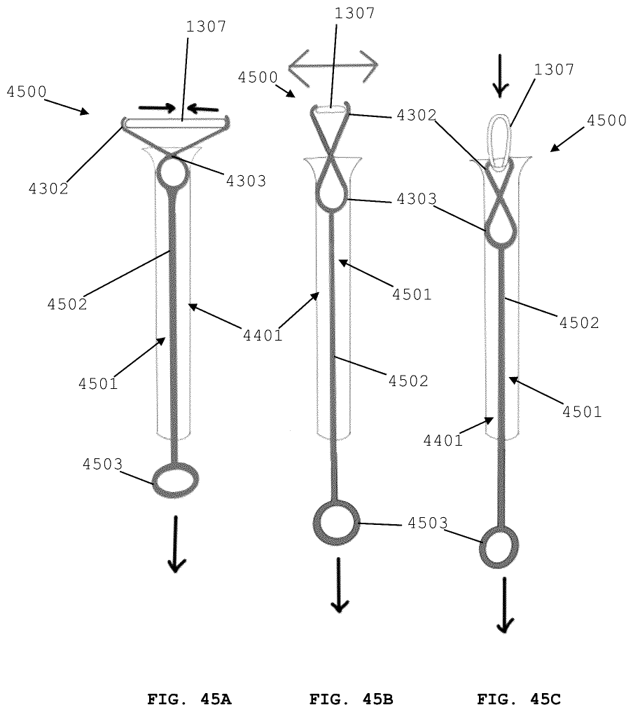

FIGS. 45A-45C shows sprung ring holder funnelled tube rigid rod introducer 4500, according to an example, which may be identical in many respects to sprung ring holder funnelled tube 4400 but in this embodiment, the drawstring 2202 may be replaced by a rigid shaft 4502 extending directly from the arm crossover section 4303 back to the opposite end of introducer tube 4501 with funnel 4401 where the surgeon may handle and therefore, push and pull the arm crossover section 4303 with as necessary. The proximal end of the rigid shaft 4502 may include a ring shaped handle 4503 for a surgeon to handle the rigid shaft 4502.

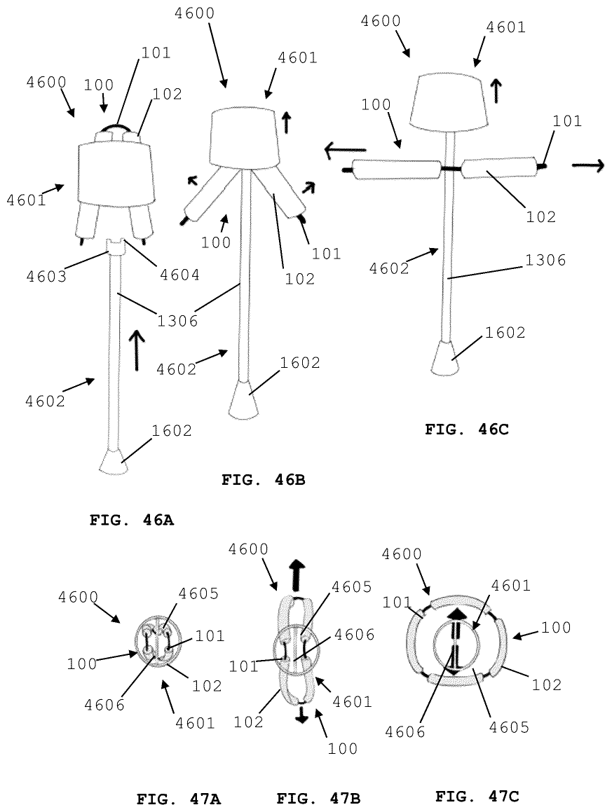

FIGS. 46A-46C and FIGS. 47A-47C shows removable cap introducer device 4600, according to an example, from a front view and a top view respectively. Removable cap introducer device 4600 includes a removable introducer cap 4601 and a removable cap introducer plunger 4602 which may include a plunger tip head 4603, plunger tip notch 4604, introducer shaft 1306 and introducer shaft handle 1602. FIGS. 46A and 47A, FIGS. 46B and 47B and FIGS. 46C and 47C show collapsible wire ring 100 in a completely compressed loaded configuration, a partially released configuration and a completely released configuration respectively.

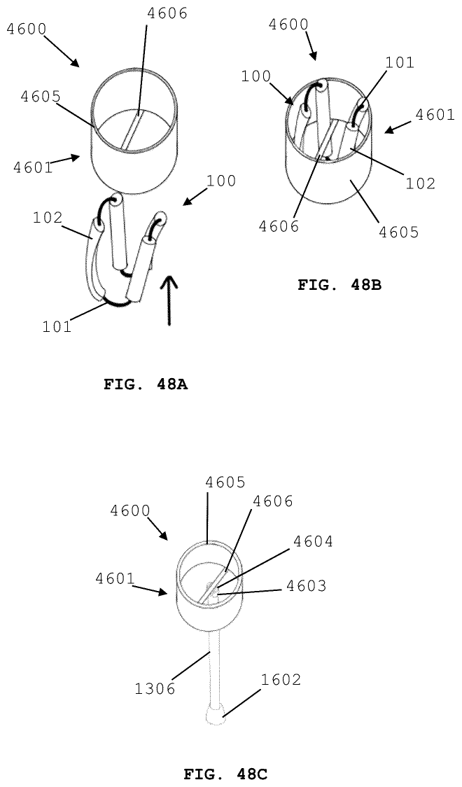

FIGS. 48A and 48B shows an isometric view of removable introducer cap 4601 which includes a cap body 4605 and a cap cross beam 4606, as it engages with collapsible wire ring 100 in isolation. Cap body 4605 may be mostly cylindrical in shape and made from a rigid or semi rigid material. Inside and at the distalmost end of cap body 4605, there may be a cap cross beam 4606 connecting from one side of cap body 4605 to the other. The cap cross beam 4606 may also be a rigid or semi rigid material. Following the compression and collapsing of collapsible wire ring 100, the two sides of the collapsed collapsible wire ring 100 may be fed up through removable introducer cap 4601 on either side of cap cross beam 4606 to be captured in a compressed configuration inside cap body 4605 (FIG. 48B). By pushing removable introducer cap 4601 upwards (e.g., distally) relative to collapsible wire ring 100, the collapsible wire ring 100 may once again be released from the removable introducer cap 4601. FIGS. 46A-46C and FIGS. 47A-47C show the introduction of removable cap introducer plunger 4602 which has a plunger tip head 4603 with a plunger tip notch 4604 that may be sized to intentionally engage with cap cross beam 4606. As introducer shaft handle 1602 may be pushed up to removable introducer cap 4601, the plunger tip notch 4604 may engage with cap cross beam 4606 and with continued pushing the removable introducer cap 4601 may move upwards (e.g., distally in a body opening) relative to collapsible wire ring 100. In this way, the collapsible wire ring 100 may be released from the removable introducer cap 4601 to open once more to its full extent (FIG. 47C) to be positioned as an anchoring element of a medical device in a body cavity. FIG. 48C shows removable cap introducer device 4600 in isometric view without collapsible wire ring 100. In some other embodiments, the introducer shaft handle 1602 may be fixedly attached to cap cross beam 4606 rather than being detachably linked as shown in removable cap introducer device 4600.

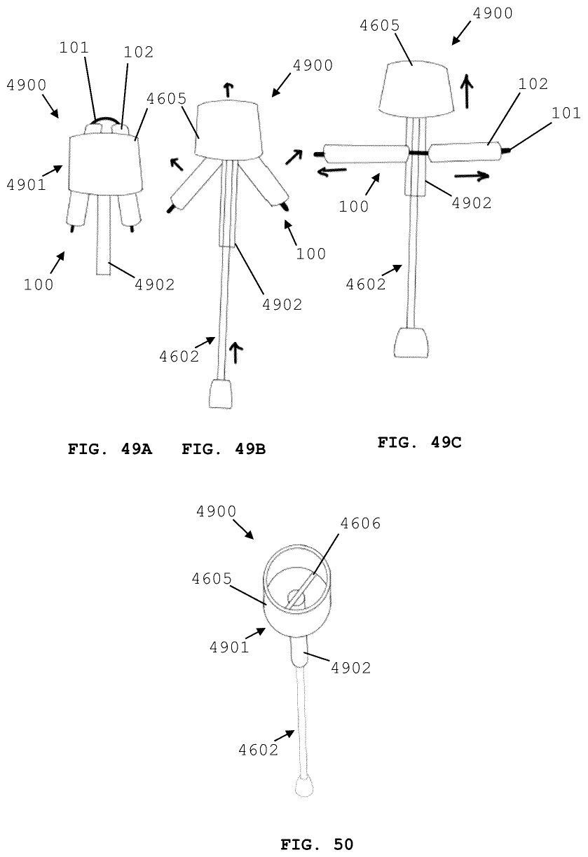

FIGS. 49A-49C and FIG. 50 show guide tube removable cap introducer 4900, according to an example, which may be almost identical to removable cap introducer device 4600. In guide tube removable cap introducer 4900, however, guide tube removable cap 4901 includes a tubular shaped extension as a guide tube 4902 for removable cap introducer plunger 4602. In embodiment removable cap introducer device 4600 removable cap, introducer plunger 4602 may not be fixed to cap cross beam 4606 and therefore, it may be difficult to approximate and align the plunger tip head 4603 to the correct location in order to engage with cap cross beam 4606 and release collapsible wire ring 100, especially when attempting this step at a distance and without clear vision during a medical procedure. Guide tube 4902 may allow for the surgeon to insert removable cap introducer plunger 4602 into a target more proximal to the surgeon and therefore, be guided to the correct position to engage removable cap introducer plunger 4602 with cap cross beam 4606 in order to release the collapsible wire ring 100. FIG. 50 shows an isometric view of guide tube removable cap introducer 4900.

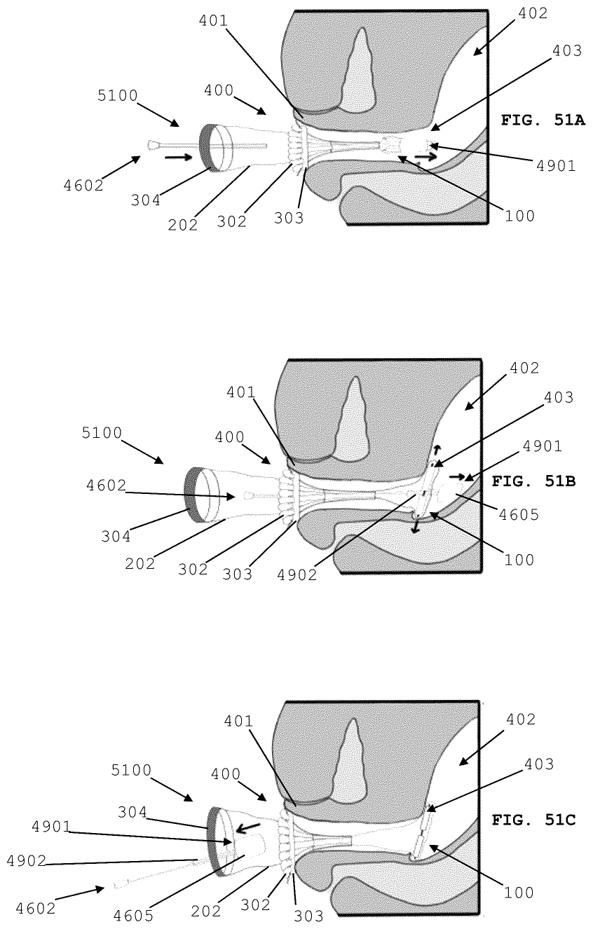

FIGS. 51A-51C show medical device with removable cap introducer 5100, according to an example, a device with sleeve 202 and guard elements guard petal 302, guard ring 303 and proximal ring 304 and also, includes all the components of aforementioned guide tube removable cap introducer 4900. Medical device with removable cap introducer 5100 is shown in a vagina 400 being pushed distally through colpotomy 403 and into body cavity 402. Sleeve 202 and collapsible wire ring 100 may be both captured in guide tube removable cap 4901 as described previously before being inserted through the vagina 400 and past colpotomy 403. Having travelled distally past colpotomy 403, the removable cap introducer plunger 4602 may be pushed forwards (FIG. 51A) to engage with guide tube removable cap 4901 and push it forwards relative to collapsible wire ring 100 and subsequently may release collapsible wire ring 100 (FIG. 51B) which may allow it to decompress and spring open. With collapsible wire ring 100 positioned in the body cavity 402 to be utilized as an anchor element for medical device with removable cap introducer 5100, the removable cap introducer plunger 4602 may be removed from the vagina (FIG. 51C)

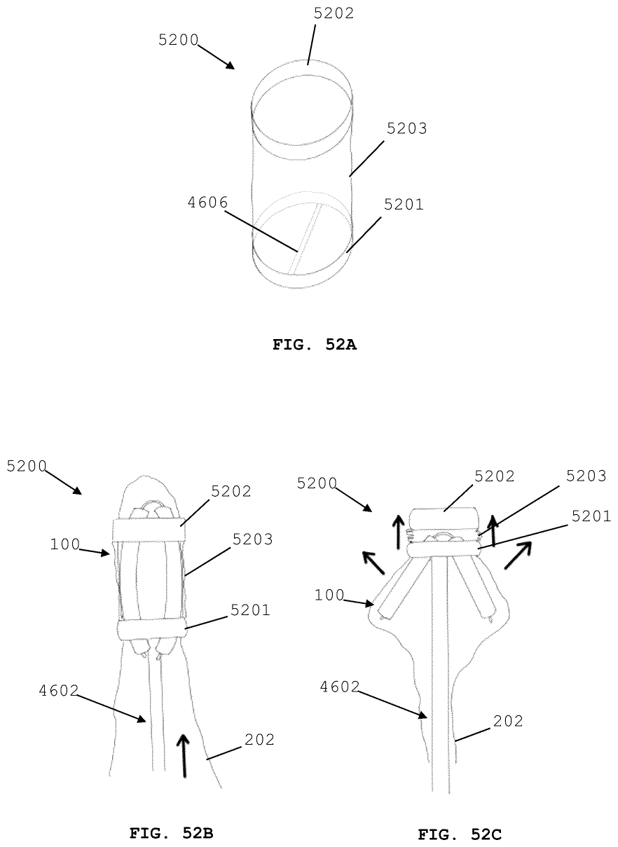

FIGS. 52A-52C shows flexible section removable cap introducer 5200, according to an example, which may be almost identical to medical device with removable cap introducer 5100 but with one difference in its construction. In flexible section removable cap introducer 5200, a large section in the middle of cap body 4605 may have been replaced with a section of flexible film material flexible middle section 5203. In this way, cap body 4605 may be replaced by a 3-component configuration including lower rigid band 5201, upper rigid band 5202 and flexible middle section 5203. Lower rigid band 5201 and upper rigid band 5202 may be two rigid bands of material connected to one another by a flexible middle section 5203 of a given length. The lower of the two bands lower rigid band 5201 may include cap cross beam 4606 which spans across the inside diameter of lower rigid band 5201 and may be made of the same rigid material from which lower rigid band 5201 is made. The flexible middle section 5203 may allow the two bands lower rigid band 5201 and upper rigid band 5202 to be approximated towards each other when required or necessary in the function of flexible section removable cap introducer 5200.

FIGS. 52B and 52C show flexible section removable cap introducer 5200 in conjunction with a collapsible wire ring 100 and sleeve 202, according to an example, that may be part of a medical device to be anchored in a body cavity. Similar to the method shown with medical device with removable cap introducer 5100 previously, the collapsed collapsible wire ring 100 may be slotted into the space either side of cap cross beam 4606 to be captured in the tubular structure created by lower rigid band 5201, upper rigid band 5202 and flexible middle section 5203. This structure may be expanded to its full extents to capture the collapsible wire ring 100 in a compressed state effectively (FIG. 52B). When it may be required to release and deploy collapsible wire ring 100, a removable cap introducer plunger 4602 may be advanced through sleeve 202 and engaged with removable introducer cap 4601. Further advancement of removable cap introducer plunger 4602 also advances the cap cross beam 4606 along with lower rigid band 5201 but upper rigid band 5202 remains in place for now as flexible middle section 5203 instead compresses with the advancement of lower rigid band 5201. With further advancement of removable cap introducer plunger 4602 and lower rigid band 5201, the flexible middle section 5203 may be compressed further until such a point when upper rigid band 5202 may be reached and no further compression of flexible middle section 5203 may be possible. At this point, further advancement of removable cap introducer plunger 4602 and lower rigid band 5201 may collide with upper rigid band 5202 and force its advancement. Together lower rigid band 5201, upper rigid band 5202 and flexible middle section 5203 may disengage with collapsible wire ring 100 past its distal end and may allow collapsible wire ring 100 to spring open to its desired circular configuration.

A practical difference between the functional use of medical device with removable cap introducer 5100 and flexible section removable cap introducer 5200 may be that the introducer components of flexible section removable cap introducer 5200 surpass the distal extent of collapsible wire ring 100 by a smaller amount than medical device with removable cap introducer 5100 which may have a fully rigid construction. In medical device with removable cap introducer 5100, the full length of cap body 4605 may surpass the distal end of the collapsible wire ring 100 while in flexible section removable cap introducer 5200 only two rigid bands lower rigid band 5201 and upper rigid band 5202 and a compressed flexible middle section 5203 must surpass the distal end of collapsible wire ring 100. This means less room may be required in a body cavity beyond the flexible section removable cap introducer 5200 in order to successfully deploy collapsible wire ring 100.

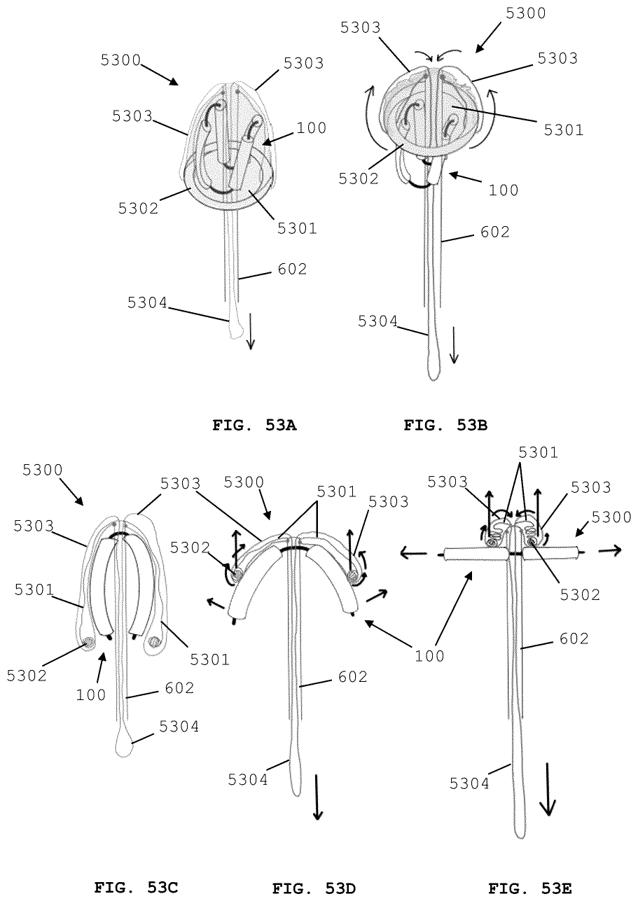

FIGS. 53A-53E show pully hood introducer 5300, according to an example. The pully hood introducer 5300 may include a sleeve hood 5301, a hood ring 5302, a pully string 5303, a pully string free end 5304 and a introducer tube 602 and is shown in conjunction with collapsible wire ring 100. Sleeve hood 5301 may be connected to the top of introducer tube 602. Sleeve hood 5301 may be constructed from a single or double layer of film material with hood ring 5302 connected to its distal end hanging down circumferentially around introducer tube 602. Pully string free end 5304 may be fed up through the centre of free end 2 606 to become pully string 5303 as it exits the other end of introducer tube 602. One side of the pully string 5303 may be passed around either side of sleeve hood 5301, wrapped around hood ring 5302 and fed back up the inside of sleeve hood 5301 where it may be fixedly connected to the tip of introducer tube 602 on the inside of sleeve hood 5301. In this way, the string may present as a looped pully string free end 5304 that that connects to the top of introducer tube 602 on either side having wrapped around the hood ring 5302 to either side first. This may create a pully effect where the pulling proximally of pully string free end 5304 may shorten the pully string 5303 on each side of sleeve hood 5301 and lift hood ring 5302 along with sleeve hood 5301 towards the top of introducer tube 602 where pully string 5303 may be ultimately connected as shown in FIG. 53B. In the configuration of the device for the introduction of collapsible wire ring 100 into a body cavity, the compressed collapsible wire ring 100 may be covered by the sleeve hood 5301 and hood ring 5302 in order to retain it in a collapsed configuration during insertion.

As further illustrated in FIGS. 53D and 53E, as the hood ring 5302 and pully string 5303 get lifted up and extends outwardly by pulling pully string free end 5304, the collapsible wire ring 100 may be released from the enclosure created by sleeve hood 5301 and upper rigid band 5202 and may be allowed to open into its desired circular configuration to serve as an anchor for a medical device in the body cavity. Following deployment of collapsible wire ring 100 into the body cavity, the device pully hood introducer 5300 may be removed.

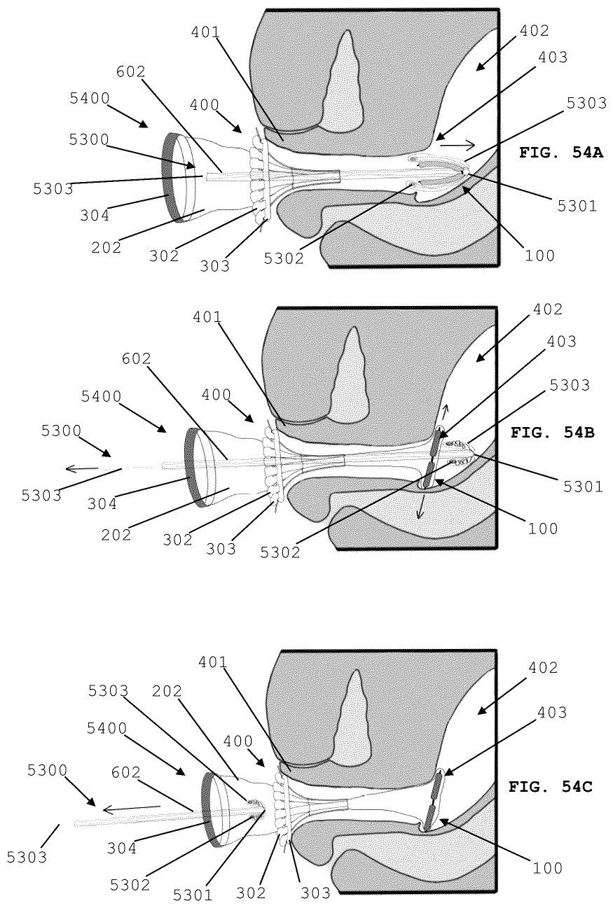

FIGS. 54A-54C show pully introducer medical device 5400, according to an example. The introducer medical device 5400 may include a sleeve 202 and guard elements guard petal 302, guard ring 303 and proximal ring 304 and also includes all the components of aforementioned pully hood introducer 5300 inserted in a vagina 400 being pushed through colpotomy 403 and into body cavity 402. Sleeve 202 and collapsible wire ring 100 may be both captured within sleeve hood 5301 as described previously before being inserted through the vagina 400 and past colpotomy 403. Having travelled past colpotomy 403 the pully string 5303 may be pulled backwards (FIG. 54A) to engage the pully which pulls hood ring 5302 and sleeve hood 5301 towards the distal end of introducer tube 602. Hood ring 5302 and sleeve hood 5301 may be pulled past the distal end of collapsible wire ring 100 and subsequently may release collapsible wire ring 100 (FIG. 54B) which may allow it to spring open to its open circular configuration. With collapsible wire ring 100 positioned in the body cavity 402 to be utilized as an anchor element for pully introducer medical device 5400, the introducer elements may be removed from the vagina (FIG. 54C).

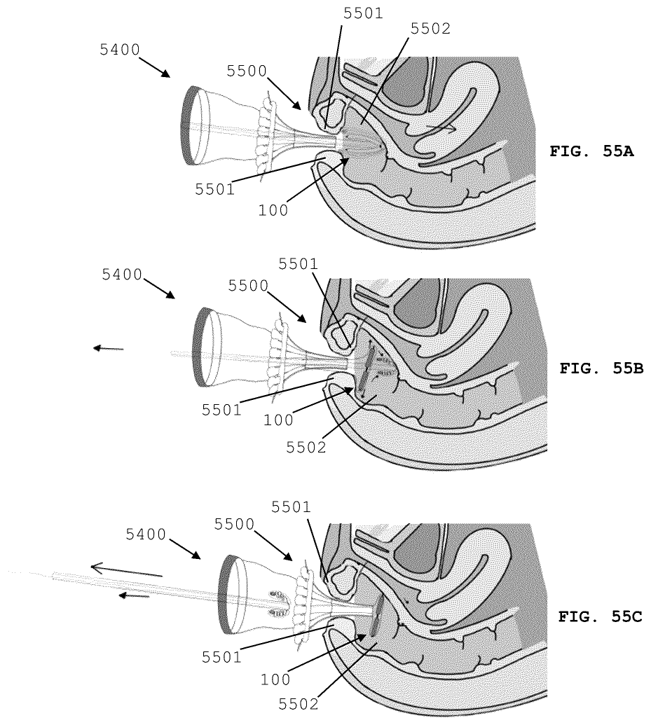

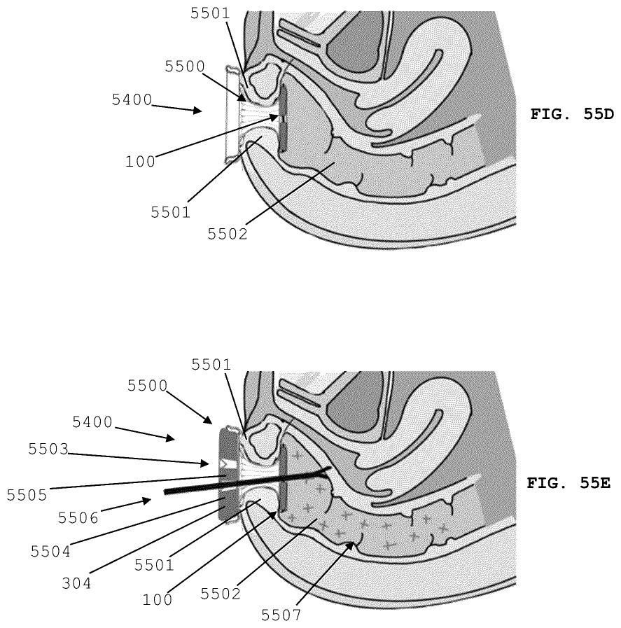

FIGS. 55A-55E illustrate pully introducer medical device 5400 being inserted into an anus 5500 through a sphincter 5501 and into the lower colon 5502 to be utilized in a transanal procedure by way of another example of natural orifice surgery. Any and all of the embodiments in this application may conceivably be used similar to that shown in FIGS. 55A-55E with pully introducer medical device 5400, through the anus 5500 and deploying and anchoring in the same way described previously.

FIG. 55D shows pully introducer medical device 5400 after deployment into the anus following deployment of collapsible wire ring 100 and removal of introducer elements of pully introducer medical device 5400. Pully introducer medical device 5400 may be configured into a retracted state following deployment in order to shorten pully introducer medical device 5400 and retract and seal the anus 5500 for more effective access for transanal medical procedures.

FIG. 55E illustrates the introduction of a valve cap 5503 that sits into proximal ring 304 of pully introducer medical device 5400 in a retracted state to create a complete seal in the anus 5500 and may allow for pneumorectum 5507 to be established in the lower colon 5502. This valve cap 5503 may include a cap housing 5504 and one or multiple valves 5505 for the introduction of one or multiple surgical instruments 5506 during a transanal surgical procedure. The valve cap 5503 or similar version of it may be used in conjunction with any or all of the embodiments described earlier in this application.

Surgical instruments introduced into the lower colon in FIG. 55E may be manually or robotically controlled instruments. Similarly, with any previously described embodiments shown inserted into the vagina, instruments that may be inserted into the body cavity through those embodiments may be manually or robotically controlled instruments.

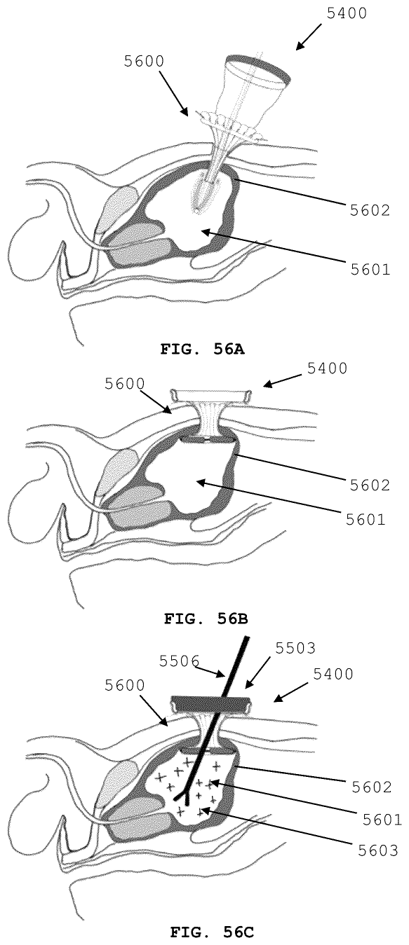

FIGS. 56A-56C shows device pully introducer medical device 5400, by way of an example embodiment from this application, inserted through an abdominal incision 5600 into the bladder 5601, a hollow organ within the abdomen, via the bladder wall 5602 (FIGS. 56A and 56B). In this way, sealed access may be granted to the surgeon to perform surgical procedures in the bladder 5601, creating a pressurized bladder 5603 (FIG. 56C) when required.

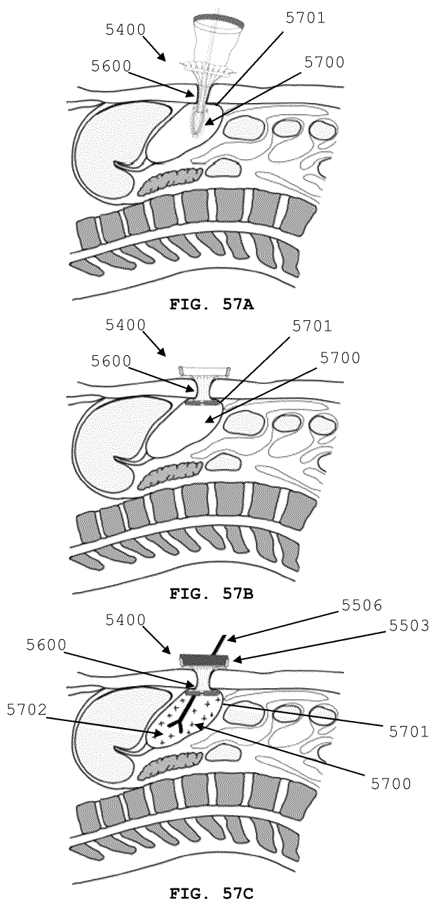

In another example, FIGS. 57A-57C shows pully introducer medical device 5400, by way of an example embodiment from this application, inserted through an abdominal incision 5600 into the stomach 5700, a hollow organ within the abdomen, via the stomach wall 5701 (FIGS. 57A and 57B). In this way, sealed access may be granted to the surgeon to perform surgical procedures in the stomach 5700, creating a pressurized stomach 5702 (FIG. 57C) when required.

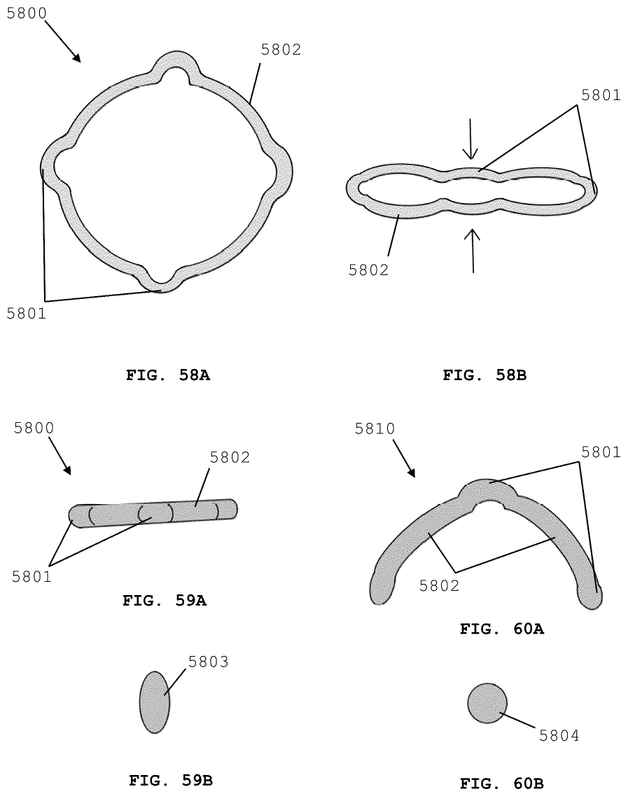

FIGS. 58A and 58B show a collapsible hinge ring 5800, according to an example. The collapsible hinge ring 5800 including one or more (e.g., four) small radius section 5801 (e.g., hinge points) and one or more (e.g., four) large radius section 5802. Each small radius section 5801 may extend as a semi-circle outwards from the collapsible hinge ring 5800. In an example, the small radius sections (e.g., all four), may be located 90 degrees apart from one another along the collapsible hinge ring. Small radius section 5801 may allow collapsible hinge ring 5800 to collapse to a tighter radius when compressed and with a reduced stress at the bend points created by small radius section 5801 compared to that of a standard ring without designated small radius sections 5801. A standard ring of equivalent size without designated small radius sections 5801 and by extension large radius section 5802, when compressed, may require more force to bend to an equivalent small radius. In many cases, with a standard ring of equivalent size without designated small radius sections 5801, it may not be possible to achieve the small radius desired. In FIG. 58B the collapsible hinge ring 5800 may be compressed at the point of two of the small radius section 5801. The two points may be 180 degrees apart from one another. In doing so, the two remaining small radius section 5801 at 90 degrees to the points of compression of collapsible hinge ring 5800 bend and due to their smaller radii the collapsible hinge ring 5800 may readily collapse to a degree not possible in a standard ring of equivalent size without designated small radius sections 5801. Other versions of this embodiment may include more or fewer small radius section 5801, and large radius section 5802 may be arranged in a non-symmetric or symmetric way and may have varying degrees of arc and radii sizes. Small radius section 5801 may be a different non-arced shape that may be similarly effective in creating a point of more effective bending of the ring under compression to its sides. The small radius section 5801 may be a semi-circular protrusion that extends radially outward from the collapsible hinge ring.

FIGS. 59A and 59B show collapsible hinge ring 5800 and its cross section (FIG. 59B) in a generally oval shape. The oval shape cross section 5803 encourages a single plane side view profile when collapsible hinge ring 5800 may be compressed. Side view of compressed collapsible hinge ring 5800 shown in FIG. 59A illustrates that the collapsible hinge ring 5800 does not curve downwards when it may be compressed as seen in FIG. 58B.

FIGS. 60A and 60B shows round cross section ring 5810 and the effect that having a circular cross section has on the round cross section ring 5810 side profile when it may be compressed. When collapsible hinge ring 5800 includes a round cross section 5804 the collapsible hinge ring 5800 bends downwards when compressed.

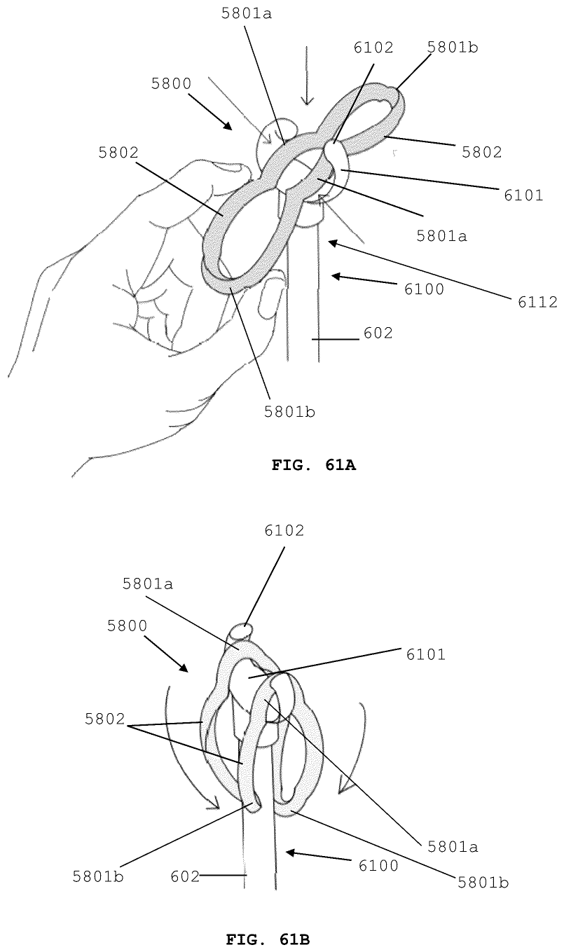

FIGS. 61A and 61B show the attachment of collapsible hinge ring 5800 to rounded head introducer 6100, according to an example. The rounded head introducer 6100 may include an introducer head 6101, outer prongs 6102 and introducer tube 602. The introducer tube 602 may be a shaft. The introducer tube 602 may include a distal end 6112 and a proximal end (not shown) at an opposite side of the introducer tube 602. The distal end 6112 of the introducer tube 602 may be coupled/connected to the introducer head 6101. The introducer head 6101 may be configured to deploy the collapsible hinge ring 5800. The introducer head 1601 may form a fork shape. The introducer 6100 may consist essentially of the shaft and the fork-shaped introducer head 1601. The introducer head 6101 may include a plurality of distally-extending prongs. For example, the introducer head 6101 may include a first prong and a second prong (e.g., outer prongs 6102) located at a distal most end of the introducer head 1601. The first prong may an inner concave surface, and the second prong includes an inner concave surface, and inner concave surfaces of the first and second prongs may face one another. The curved/concaved inner face may for example match a curvature of an outer surface of the collapsible hinge ring 5800 (or any of the distal anchoring member described in this application).

The configuration of collapsible hinge ring 5800 shown in FIG. 58B may be placed into rounded introducer head 6101 between outer prongs 6102. For example, two hinge points 5801a located 180 degrees apart may be compressed and placed within outer prongs 6102. The outer prongs 6102 may include a curved inward face to receive the curved cross section of the outside of the collapsible hinge ring 5800. The outward force of the collapsible hinge ring 5800 trying to spring open pushes out against outer prongs 6102 and the collapsible hinge ring 5800 may be captured. Next, as shown in FIG. 61B, The collapsible hinge ring 5800 may then be folded again in the downward direction at two hinge points 5801b located 180 degrees apart. The two hinge points 5801b may be folded and pulled down in a proximal direction to a proximal end of introducer tube 602. The hinge points 5801a, 5801b may bends readily to a smaller radius that may be possible in a standard ring of equivalent size without designated small radius sections 5801.

In another example, the introducer head 6101 may have a plurality of prongs; a controllable hood; a clasp ring grasper; a removable cap; or a pulley arrangement as described early in the application. The collapsible hinge ring 5800 may have a diameter ranging from 2 cm to 10 cm. In another example, the collapsible hinge ring 5800 may be partially exposed nitinol ring; a concertina ring; a severed ring; a spring ring; a sliding ring; a concertina section ring; or a notched ring as described earlier in the application. The collapsible hinge ring 5800 and corresponding medical device that may be described in more detail below may be utilized in an opening of a patient. The openings may include, but are not limited to vagina, anus, colpotomy, or esophagus. The introducer tube 602 may extend from a proximal end (not shown) to a distal end 6112. The length from the proximal end to the distal end 6112 of tube 602 may be between 5 cm to 30 cm. In an example, when the introducer tube 602 is being utilized in a vagina, it may preferably be 5 cm to 20 cm. In an example, when the introducer tube 602 is being utilized in a vagina, it may preferably be 5 cm to 20 cm. In an example, when the introducer tube 602 is being utilized in a throat, it may preferably be 20 cm to 30 cm. In an example, when the introducer tube 602 is being utilized in an anal cavity, it may preferably be 5 cm plus or minus 2 cm.

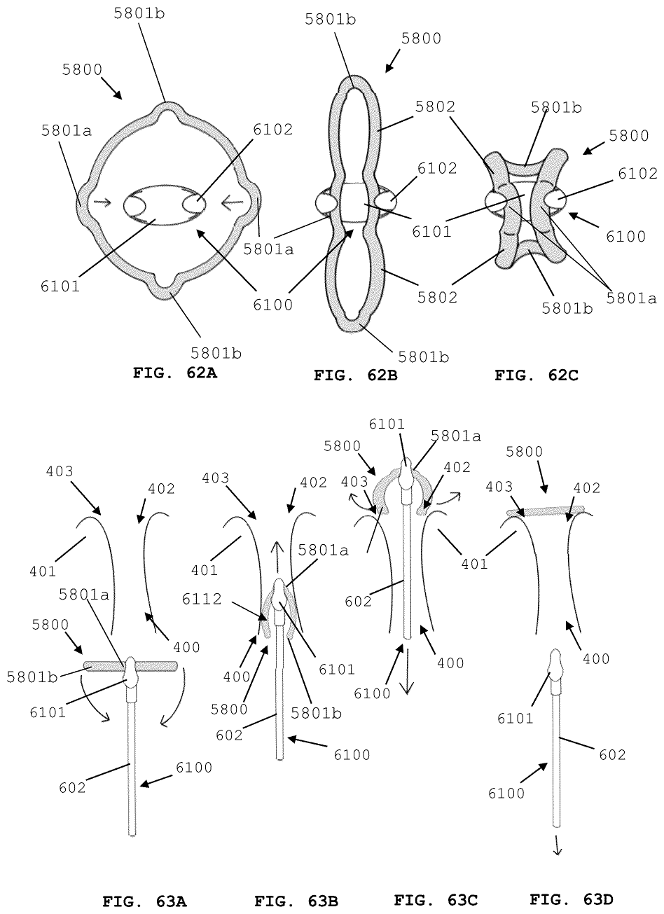

FIGS. 62A-62C show the steps described in FIGS. 61A and 61B from a top view. The small profile shown in FIG. 62C may allow for introduction into an incision or body opening with less resistance.

FIGS. 63A-63D may show the step-by-step introduction of a collapsible hinge ring 5800 using rounded head introducer 6100. FIG. 63A shows the collapsible hinge ring 5800 compressed in one plane using the first hinge points 5801a and loaded into rounded head introducer 6100 in preparation for insertion into a vagina 400. FIG. 63B shows that as collapsible hinge ring 5800 is advanced into the vagina, the counter forces acting towards collapsible hinge ring 5800 from vaginal wall 401 forces collapsible hinge ring 5800 to bend in the second plane downwards using the second set hinge points 5801b. FIG. 63C shows the collapsible hinge ring 5800 and rounded introducer head 6101 passing through the colpotomy 403 into the body cavity 402, at which point the collapsible hinge ring 5800 may be allowed to release itself from the second bend forced upon it by vaginal wall 401 due to reaching the space available in body cavity 402. In FIG. 63D the rounded head introducer 6100 may be pulled backwards, proximally, out of the vagina 400. As it is passed back through colpotomy 403, the now semi-unfolded collapsible hinge ring 5800 comes into contact with the back of colpotomy 403, owing to being wider than the colpotomy 403 opening, and this small force may encourage the collapsible hinge ring 5800 to pop out of the rounded introducer head 6101 to be deployed into the body cavity in an open configuration to serve as an anchor ring for a medical device.

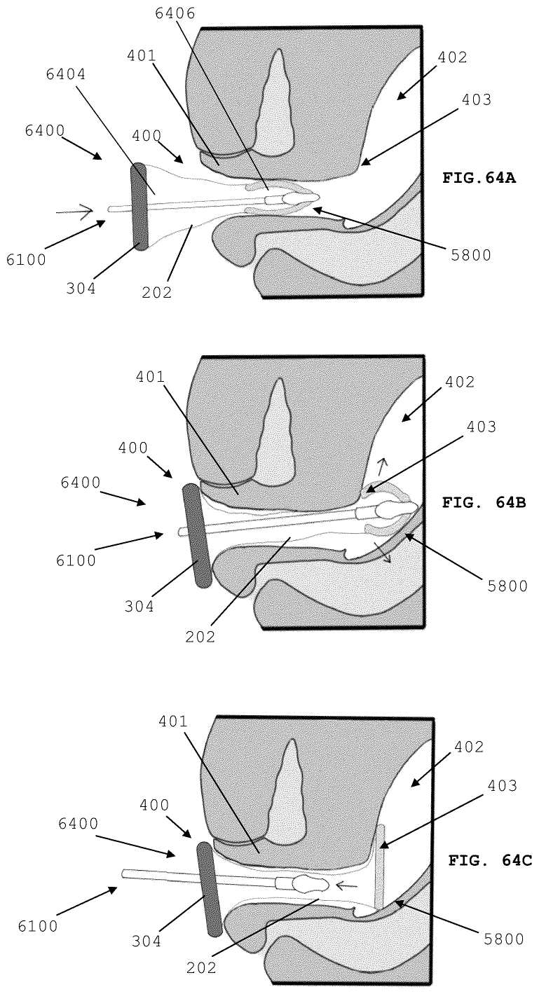

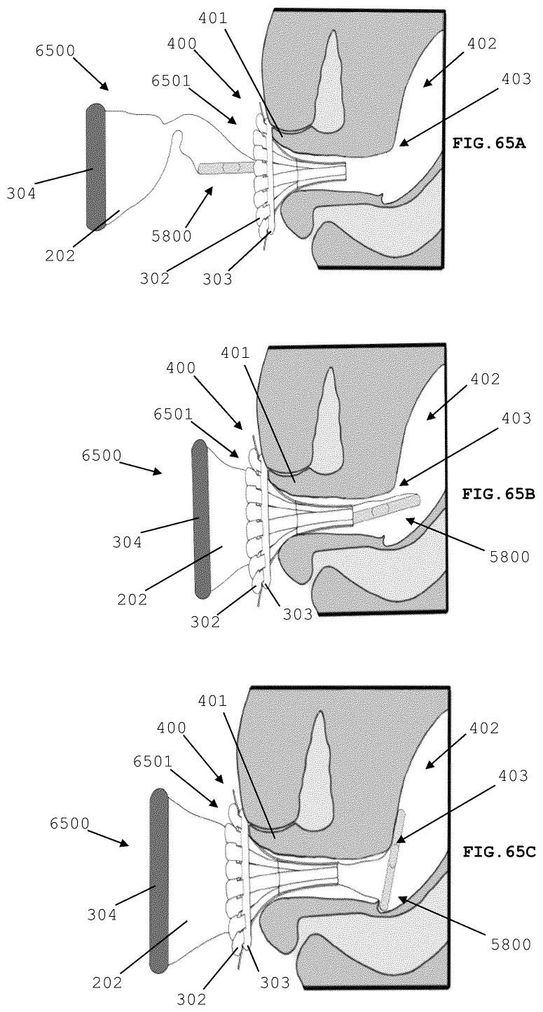

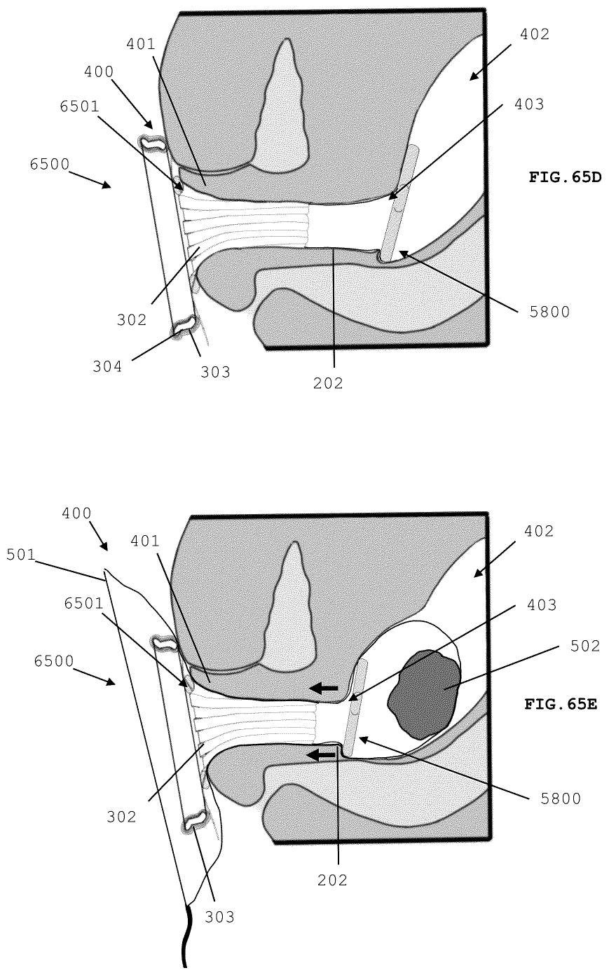

Examining the method of deploying the collapsible hinge ring 5800, for example as shown in FIGS. 63A-63D, the method may include first compressing a first portion and a second portion of the collapsible hinge ring 5800 (e.g., at hinge points 5801a located opposite and/or 180 degrees apart from one another). FIGS. 63A-63D may depict how to deploy collapsible hinge ring 5800 as well as all of the various collapsible rings described herein in this disclosure. The method may next include positioning the first portion and second portion of collapsible hinge ring 5800 (e.g., at hinge points 5801a) into the introducer head 6101 (e.g., in-between the introducer head 6101 outer prongs 6102 as depicted in FIG. 62B). The introducer head 6101 may be fork-shaped. Next, the collapsible hinge ring 5800 may be folded at a third portion and fourth portion (e.g., at hinge points 5801b located 180 degrees apart from one another) in a proximal direction down introducer tube 602 towards the proximal end of the introducer tube. A first portion (e.g., hinge point 5801A) may be ninety degrees apart from a third portion (e.g., hinge point 5801B). This may either be done by hand (e.g., as shown in FIG. 61A), or by the walls of a patient opening (e.g., as shown in FIG. 63B). Next, the distal end 6112 of the introducer tube 602 may be inserted into a body cavity. The body cavity may include, but is not limited to, a vagina, colpotomy, an anus, an incision, or an esophagus. Next, the collapsible hinge ring 5800 may be released within the body cavity. The collapsible hinge ring 5800 may decompress upon release from the introducer head 6101 and expands radially to a natural shape. Releasing the collapsible distal anchor member within the body cavity may include the distal anchor member sliding off the fork-shaped distal end portion and expanding radially in the body cavity. Next, the introducer tube 602 may be extracted from the body cavity. The steps may further include expanding an outer wall of the sleeve 202 so that a cavity all of the body cavity retracts; inserting one or more medical instruments through the device and into a body cavity, and performing a surgical procedure using at least one or more surgical instruments. For example, the collapsible hinge ring 5800 may be connected to a sleeve 202, proximal ring 304, and a guard (e.g., guard petals 302) as illustrated in FIGS. 64A-64C, or the collapsible hinge ring 5800 may be connected to a sleeve 202, a proximal ring 304, and a guard (e.g., guard petals 302) as illustrated in FIGS. 65A-65E, but now shown in FIGS. 63A-63D. The sleeve 202 may be slideably or fixedly coupled to the proximal ring 304 and any of the distal rings described in this application (e.g., collapsible hinge ring 5800).

FIGS. 64A-64C show medical retractor device 6400, according to an example. The medical retractor device 6400 may include a sleeve 202 with a proximal end 6404 and a distal end 6406, a proximal ring 304 coupled to the proximal end 6404 of the sleeve 202, and a collapsible hinge ring 5800 coupled to the distal end 6406 of sleeve 202. As depicted in FIGS. 65A-65E, in some embodiments of the medical retractor device 6400 may include a guard device coupled to the sleeve 202. The proximal ring 304, sleeve 202, and collapsible hinge ring 5800 may for a lumen for access into the opening in a patient. Medical retractor device 6400 may further include a rounded head introducer 6100 configured to deploy the collapsible hinge ring 5800. The rounded heat introducer 6100 may be configured to attach and detach from the collapsible hinge ring 5800. The same steps described for FIGS. 63A-63D may repeated in FIGS. 64A-64C in order to deploy collapsible hinge ring 5800 into the body cavity. Further, although not pictured, the steps described for FIG. 63A-63D may be repeated to deploy the collapsible hinge ring 5800 into a body cavity using an introducer 6100 (not shown in FIGS. 65A-65E).

FIGS. 65A-65E show separate guard device 6500, according to an example, which includes a medical retractor device 6400 and separate guard 6501 where medical retractor device 6400 may be inserted through a separate guard 6501 already placed inside the vagina 400 between the vaginal wall 401. The separation of the separate guard 6501 from the sleeve 202 as is previously described in other embodiments offers more flexibility and less restriction to collapsible hinge ring 5800 during insertion into the body cavity 402, particularly when the length of the vagina 400 may be longer that average. In another example, the separate guard 6501 may be coupled to sleeve 202 (e.g., by pockets in a sleeve). The separate guard 6501 may include a plurality of long-skirt petals (e.g., as depicted in FIGS. 38A through 39C) or an outer casing assembly. Collapsible hinge ring 5800 may be inserted by hand or by using rounded head introducer 6100 depending on the length of the vagina 400 or other natural opening and reach of the surgeon user.

FIG. 65D shows separate guard device 6500 in a retracted configuration. By flipping proximal ring 304 inwards the sleeve 202 length may effectively shorten by bringing collapsible hinge ring 5800 towards 304. In doing so, sleeve 202 may be forced radially outwards against the vaginal wall 401, effectively retracting it to a wider opening. The separate guard 6501 extends into the vagina 400 offering protection from sharp instruments.

FIG. 65E shows separate guard device 6500 inserted into the inside of containment bag 501 situated in the body cavity 402 and vagina 400, according to an example. Opening of containment bag 501 may be extended out through colpotomy 403 and further through the vagina 400 to present its opening to the surgeon during a medical procedure. Containment bag 501 contains a specimen 502 for removal, which could be any tissue specimen or organ to be removed from the body cavity 402 depending on the incision or natural orifice used to gain access. The insertion of separate guard device 6500 including collapsible hinge ring 5800 provides the surgeon with access to specimen 502 in order to carry out necessary surgical techniques used to reduce its size and remove it, such as by cutting with scalpel through the retracted vaginal wall 401. In instances where sharp instruments may be used through the vagina 400 to reduce specimen 502 for removal the guard petals 302 may protect the containment bag 501 and the vagina 400 from cutting or damage by sharp instruments.

Further indicated by the arrows in FIG. 65E is the effective shortening of the vaginal canal when the separate guard device 6500 is retracted by flipping proximal ring 304 and bringing proximal ring 304 closer to collapsible hinge ring 5800. This same practice is true for any and all embodiments described in this application with any form of anchoring ring system incorporated with a access medical device.

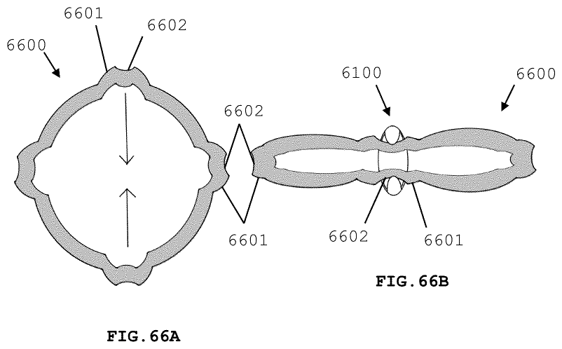

FIGS. 66A and 66B show another ring embodiment hinged ring version 2 6600, according to an example, similar to collapsible hinge ring 5800, but with the addition of notch within small radii section 6602. This notch within small radii section 6602 may be built into an existing small radii section 6601. Without compromising the bending benefits of small radii section 6601, the notch within small radii section 6602 offers a profile more amenable to attachment of the rounded introducer head 6101 for introduction into a body cavity. The notch within small radii section 6602 may offer rounded introducer head 6101 a better grip on hinged ring version 2 6600 to minimise sliding out of position during the insertion process.

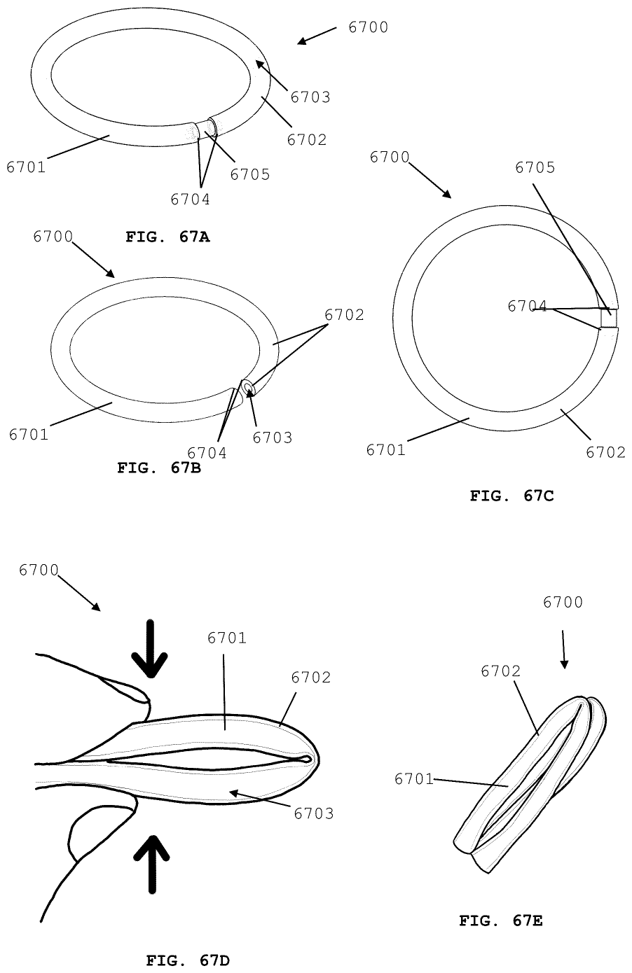

FIGS. 67A-67E shows a tubed distal ring 6700 according to another example of an anchoring ring that may be used in a sleeved retractor medical device for the purposes of establishing access to a body cavity through an incision or a natural orifice. FIG. 67A shows the tubed distal ring 6700 including a tubed element 6701, a tube wall 6702 and a tube inner channel 6703, and a connector element 6705 (e.g., tube connector). Tube element 6701 may be made from a semi rigid/flexible material, for example rubber, PVC, polyurethane etc. Tube element 6701 may be configured into a mostly circular shape (FIG. 67B) and its ends 6704 may be connected to each other by connector element 6705 (FIGS. 67A & 67B). Tube ends 6704 may be connected to each other in a mostly circular shape by any fixation means, for example glueing, welding etc. in order to achieve the somewhat circular ring shape seen in FIG. 67C.

The mechanics of tubed distal ring 6700 may allow for the ring to collapse easily when compressed by a user. When a bending force is applied to tube element 6701, the two opposing tube walls 6702 either side of the tube inner channel 6703 at that point may compress inwards into the hollow space of the tube inner channel 6703 creating a local compression (FIG. 67D). This local compression of the tube element 6701 may allow the tubed distal ring 6700 to collapse more easily and to a smaller radius than an otherwise completely solid distal ring. In this way, the tubed distal ring may compress overall to a smaller size when folded twice in order to more easily insert through an incision or natural orifice into a body cavity during surgery (FIG. 67E).

What is claimed is:

1. An introducer for inserting a medical access device into an opening of a patient, the medical access device including a sleeve portion including a proximal end and a distal end; a proximal ring coupled to a proximal end of the sleeve portion; a collapsible distal anchor member coupled to the distal end of the sleeve portion;
wherein the sleeve portion, proximal ring, and distal anchor member form a lumen for access into the opening of the patient, the introducer comprising:
a shaft having a proximal end and a distal end; and
an introducer head at the distal end of the shaft, the introducer head including a plurality of distally-extending prongs.

2. The introducer of claim 1, wherein the distally-extending prongs include a first prong and a second prong located at a distal most end of the introducer head.

3. The introducer of claim 2, wherein the first prong includes an inner concave surface, and the second prong includes an inner concave surface, and inner concave surfaces of the first and second prongs face one another.

4. The introducer of claim 2, wherein the introducer head includes a third prong in between the first prong and the second prong at the distal most end of the introducer head.

5. The introducer of claim 2, wherein the first prong has a greater length than the second prong, and both the first prong and second prong are curved in a same direction.

6. The introducer of claim 1, wherein the plurality of distally-extending prongs of the introducer head form a fork shape.

7. The introducer of claim 6, wherein the introducer consists essentially of the shaft and the fork-shaped introducer head.

8. A medical access device comprising an access device for access through an opening of a patient, and an introducer for the access device,
the access device including:
a sleeve portion including a proximal end and a distal end;
a proximal ring coupled to a proximal end of the sleeve portion; and
a collapsible distal anchor member, coupled to the distal end of the sleeve portion; wherein the sleeve portion, proximal ring, and distal anchor member form a lumen for access through an opening of a patient, and
the introducer including:
a shaft having a proximal end and a distal end; and
a fork-shaped distal end portion at the distal end of the shaft configured to receive and deploy the collapsible distal anchor member.

9. The medical access device of claim 8, wherein the collapsible distal anchor member includes a plurality of hinge points.

10. The medical access device of claim 9, wherein each of the plurality of hinge points is a semi-circular protrusion that extends radially outwards, wherein a radius of the semi-circular protrusion is smaller than a radius of the collapsible distal anchor member.

11. The medical access device of claim 10, wherein the plurality of hinge points includes a first hinge point, a second hinge point, a third hinge point, and a fourth hinge point, wherein the first hinge point and second hinge point are one hundred and eighty degrees apart; the third hinge point and fourth hinge point are one hundred and eighty degrees apart; and the first hinge point and third hinge point are ninety degrees apart.

12. The medical access device of claim 8, wherein the collapsible distal anchor member is one of a: partially exposed nitinol ring; a concertina ring; a severed ring; a spring ring; a sliding ring; a concertina section ring; or a notched ring.

13. The medical access device of claim 8, wherein the fork-shaped distal end portion includes an introducer head with a first prong and a second prong located at a distal most end of the introducer head.

14. The medical access device of claim 13, wherein the first prong includes an inner concave surface, and the second prong includes an inner concave surface, and inner concave surfaces of the first and second prongs match a curvature of an outer surface of the collapsible distal anchor member.

15. The medical access device of claim 8, wherein the shaft has a length from the proximal end to the distal end of 5 cm to 30 cm.

16. The medical access device of claim 8, the opening of the patient includes a body opening.

17. A method of inserting an access device into a body cavity, the access device including a sleeve portion including a proximal end and a distal end; a proximal ring coupled to a proximal end of the sleeve portion; a collapsible distal anchor member coupled to the distal end of the sleeve portion; wherein the sleeve portion, proximal ring, and the collapsible distal anchor member form a lumen for access into the body cavity, the method comprising:
compressing a first portion and a second portion of the collapsible distal anchor member, wherein the first portion and second portion of the collapsible distal anchor member are located opposite one another;
positioning the first portion and the second portion of the collapsible distal anchor member into an introducer, the introducer including:
a shaft having a proximal end and a distal end; and
a fork-shaped distal end portion configured for securing and deploying the collapsible distal anchor member;
folding a third portion and a fourth portion of collapsible distal anchor member down the shaft of the introducer towards the proximal end;
inserting the distal end portion of the introducer into the body cavity;
releasing the collapsible distal anchor member within the body cavity by retracting the introducer; and
extracting the introducer from the body cavity.

18. The method of claim 17, further including, upon extracting the introducer from the body cavity:
expanding an outer wall of the sleeve portion so that a cavity wall of the body cavity retracts; and
inserting one or more medical instruments through the access device and into the body cavity.

19. The method of claim 17, wherein positioning the collapsible distal anchor member into an introducer includes placing the compressed first portion and the second portion of the collapsible distal anchor member within prongs of the distal end portion of the introducer.

20. The method of claim 17, wherein releasing the collapsible distal anchor member within the body cavity includes the distal anchor member sliding off the fork-shaped distal end portion and expanding radially in the body cavity.
